SEQUENCE LISTING (64 sequences)

SEQ ID NO: 1 - DNA, 37 bp, artificial
  PCR primer
gacgacgaattcaagcttatggtgttttgatcatttt

SEQ ID NO: 2 - DNA, 31 bp, artificial
  PCR primer
gacgacgaattcatacatcgcatcgacaagg

SEQ ID NO: 3 - DNA, 1407 bp, Homo sapiens
  sig_peptide (1)..(57)
atggagtttgtgctgagctgggttttccttgttgctatattaaaaggtgtccagtgtgagggtcagctggtgcaatctgggggaggcttggtacatcctggggggtccctgagactctcctgtgcaggctctggattcaccttcagtagctatggtatgcactgggttcgccaggctccaggaaaaggtctggagtgggtatcaggtattggtactggtggtggcacatactctacagactccgtgaagggccgattcaccatctccagagacaatgccaagaactccttgtatcttcaaatgaacagcctgagagccgaggacatggctgtgtattactgtgcaagaggagattactatggttcggggagtttctttgactgctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 4 - DNA, 27 bp, artificial
  PCR primer
gacggatccaccatggagtttgtgctg

SEQ ID NO: 5 - DNA, 27 bp, artificial
  PCR primer
gacctcgagtcatttacccggggacag

SEQ ID NO: 6 - DNA, 708 bp, Homo sapiens
  sig_peptide (1)..(66)
atggacatgagggtcctcgctcagctcctggggctcctgctgctctgtttcccaggtgccagatgtgacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttaccctcccacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 7 - DNA, 28 bp, artificial
  PCR primer
gacggatccaccatggacatgagggtcc

SEQ ID NO: 8 - DNA, 28 bp, artificial
  PCR primer
gacctcgagctaacactctcccctgttg

SEQ ID NO: 9 - DNA, 29 bp, artificial
  PCR primer
gacggatccaccatgaaggcgatcctctc

SEQ ID NO: 10 - DNA, 35 bp, artificial
  PCR primer
ctgggtcatctggatgtcacgcttcaccaaagggc

SEQ ID NO: 11 - DNA, 35 bp, artificial
  PCR primer
gccctttggtgaagcgtgacatccagatgacccag

SEQ ID NO: 12 - protein, 290 aa, artificial
  Fusion of cellulose binding domain from Meripilus gigantus and kappa light chain from H. sapiens
MKAILSLAAALLSAAPAFSTAVWGQCGGIGFSGDTTCTASTCVKVNDYYSQCQPGASAPTSTASAPGPSACPLVKRDIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1401 bp, Homo sapiens
  sig_peptide (1)..(57)
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 14 - DNA, 20 bp, artificial
  PCR primer
gcaagggatgccatgcttgg

SEQ ID NO: 15 - DNA, 39 bp, artificial
  PCR primer
gactccaacagctggacctcggccaaagcaggtgctgcg

SEQ ID NO: 16 - DNA, 39 bp, artificial
  PCR primer
cgcagcacctgctttggccgaggtccagctgttggagtc

SEQ ID NO: 17 - DNA, 38 bp, artificial
  PCR primer
caacagctggacctcacactggacaccttttaatatag

SEQ ID NO: 18 - protein, 467 aa, artificial
  Fusion of the IgG1 signal with the IgG2 heavy chain
MEFVLSWVFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - DNA, 705 bp, Homo sapiens
  sig_peptide (1)..(60)
atggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 20 - DNA, 40 bp, artificial
  PCR primer
gactgcgtcaagacgatctcacatctggcacctgggaaac

SEQ ID NO: 21 - DNA, 40 bp, artificial
  PCR primer
gtttcccaggtgccagatgtgagatcgtcttgacgcagtc

SEQ ID NO: 22 - DNA, 28 bp, artificial
  PCR primer
gacctcgagttagcactcgcccctgttg

SEQ ID NO: 23 - protein, 237 aa, artificial
  Fusion of the IgG1 kapp signal with the IgG2 kappa chain
MDMRVLAQLLGLLLLCFPGARCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - DNA, 35 bp, artificial
  PCR primer
cctgaattcacgcgcgccaacatgtcttccaagtc

SEQ ID NO: 25 - DNA, 31 bp, artificial
  PCR primer
gttctcgagctacttattgcgcaccaacacg

SEQ ID NO: 26 - DNA, 18 bp, artificial
  PCR primer
accatggcggcactctgc

SEQ ID NO: 27 - DNA, 18 bp, artificial
  PCR primer
gagccgtaggggaagtcc

SEQ ID NO: 28 - DNA, 19 bp, artificial
  PCR primer
cttcagactgaacctcgcc

SEQ ID NO: 29 - DNA, 20 bp, artificial
  PCR primer
gactcggtccgtacattgcc

SEQ ID NO: 30 - DNA, 38 bp, artificial
  Oligonucleotide for correction of the defect pyrG gene
cctacggctccgagagaggccttttgatccttgcggag

SEQ ID NO: 31 - DNA, 20 bp, artificial
  PCR primer
gagttagtagttggacatcc

SEQ ID NO: 32 - DNA, 33 bp, artificial
  PCR primer
ctggatgacatgtatcatgaaggtatgtgaatc

SEQ ID NO: 33 - DNA, 32 bp, artificial
  PCR primer
tgttgtctcgagggagagggagaaggagaggg

SEQ ID NO: 34 - DNA, 32 bp, artificial
  PCR primer
cccgcactcgagctgaaatcgacgtggaattc

SEQ ID NO: 35 - DNA, 37 bp, artificial
  PCR primer
gttattttgcggccgccctatccaacgttggacgatc

SEQ ID NO: 36 - DNA, 3374 bp, Aspergillus oryzae
gaattctgtagctgctctatttctattactgtgtattttccttcctctcttaggttgtggaggttgagatagaggttactcttctagatacgggtccaggtccccggtaaataaacgtttcgagggacacaggttagctagtagaatgggaacatctcgatagtatctcagcatgcaagggagagaagttgtacaaaccgcggggcaataagttggcactctaagaatggtgtttctctctcttttggggtcgatttaccgggcgtcggaaacagtgtcgagtgtaaatgcatacatataacactagaatggtagaatgtctaggtatcctgactattatagcttcagatattgccaagtaagaccggaagcgtgactttatatcgcctgacgaaggatagaagagcctacgctaaagcaaagttgttctctcaagtagccctaggtgggaattgctttcgatcgcaaatcaatttgagaaatgccaccaccaccaaatgtaggtcttcaaaactgaaaatcgatcttgattaagcctcgatggatgttcagacttatatatcgttgtagtttgcgtgggatgtacaaccaggtatctacgtcgtggattctgtagggaaagtagtaatgtcttgaggcgactggtaatgtgagtatgcaaggagattgcgagaccggaccacaattactatatcatggtctacccatgatctagcctccgcccagctcactatagatgttcttcagacaggggacggcgaattcacgggcgaaatcaatttgtggctgcatcctcatgtcttgaaacaagcagtgtgattggaagtggtggcgtcttggctccagccagctgcatgactatgagaaaatagtccaactagtcttggcttgcagaatgacttccggaaatttaacctggtggctgctctcagtattggctgacgctccgcacctctaattgtgcttcaggtcagtcaaagatgttttgaaccaccaacgagccgaacccttccgccgctacaatactcgggttattataaaacgcatcgtggttcatcgtcccagctataggattattttcacatcagcaaacgaagtcgaagcaagatggtgtctttctcctcttgtctccgggccttagccctcggatcctcagttctcgcggtccaacctgtccttagacaggcgactggtctagatacctggctgagcacagaagcaaatttttcccgccaggcaatcttgaataatatcggcgcagatggccagtcggcgcagggcgcaagtccaggggtggtgattgccagccctagcaaaagtgacccagattgtacgtgctctggccttttgcaatccttcatttgacttactggccgtagatttctatacctggacccgtgactcgggtctcgtcatgaaaaccctggtcgatctgttcagaggcggagatgccgatcttctccctatcatcgaggagttcattagctcccaggctcggatccaaggcatctcaaacccttctggtgctctttccagtgggggtctgggcgagcctaagttcaatgtcgacgagacagcatttaccggcgcatggggtcggccgcagcgtgacggaccagctttgcgcgcgaccgctatgatctcgtttggagaatggctagttgtaagttccatccgtctttcagaatgggcgtagttaccttataggaaaatagtcatacaagcatagcgacggacctggtatggcctgttgttaggaatgatctatcctatgtagctcagtattggagccaatccgggttcggtgagcttctagagaaatggcccgcagctatgcagtagctaacttccacagatctctgggaggaagtccaaggcacatcattctttactgttgcagtttctcatcgcgctttggtagaaggtagcagcttcgcaaagactgtcggttcctcgtgcccctattgtgactcgcaagcgccccaagtccgatgttatttacaatccttctggaccgggagttacatccaggccaatttcggtggcgggcgatcgggcaaagacatcaacactgtcctgggtagtatccacacgttcgatcctcaagcgacgtgtgatgatgctaccttccagccctgttcggcgcgagcattggccaaccataaggtagtaacggactcgttccgatcaatctatgccatcaactccggtcgtgctgagaatcaagctgttgctgttggccgctaccccgaagacagctattacaacgggaatccttggttcctgaccaccctggccgccgcagagcagttgtatgacgcgttgtaccagtgggataaaattggatcattggccatcacggacgtttctttgccattcttcaaagctctttacagttctgccgcgacagggacctacgcatcgtccacgacggtgtataaggatattgtctcagccgtcaaggcctatgcagacggatacgtgcagatcgtcgtacgtcaagttccctcttcactctcgttaatggttagggttgctaatgaccgcagcaaacctacgctgcatccaccggctccatggccgagcaatataccaagacggacgggagtcagacctccgcccgggatcttacctggtcgtacgctgcacttctcacggccaacaaccgacgaaacgcggtcgttcctgcaccatggggcgagaccgctgccaccagcattccgtcagcttgctctacgacttccgcctcgggcacctacagcagcgtggttatcacatcctggccgaccattagcggatacccaggcgcgccagacagcccctgccaggtgccgacgactgtgtcggtgaccttcgcggtgaaagctactacggtctacggtgagtctatcaagatcgtcgggtcgatctctcagctcgggagctggaatcctagcagcgcgaccgcattgaacgcggacagctacactactgacaaccccttgtggacgggaacaataaacttgcctgctggacagtcgttcgagtataagtttattcgcgttcagaacggggcggttacgtgggagagtgaccccaaccggaaatataccgttccttcgacttgcggggtgaaaagtgctgtgcagagcgatgtttggcggtgatcatcatgtcccgatgaagaggaggaattggtacgtggtattggtgatgtggcgctgtcatattcagtatatatgttcgattctgtgaaatccgaaggcagagagaccaagagagggcccacctattcgtgtgtaatagacaagataatgttagtgtcctatgaaatcccatgatataaatttcgagaagcaggacctacacagatgtatccgattatccttggatcc

SEQ ID NO: 37 - DNA, 29 bp, artificial
  PCR primer
ggaagatctgctgctctatttctattact

SEQ ID NO: 38 - DNA, 30 bp, artificial
  PCR primer
cccaagcttctgggacgatgaaccacgatg

SEQ ID NO: 39 - DNA, 30 bp, artificial
  PCR primer
cccaagcttggatcattggccatcacggac

SEQ ID NO: 40 - DNA, 29 bp, artificial
  PCR primer
cgcgtcgacggatccaaggataatcggat

SEQ ID NO: 41 - DNA, 21 bp, artificial
  PCR primer
gttggccaatgctcgcgccga

SEQ ID NO: 42 - DNA, 21 bp, artificial
  PCR primer
gccctcggatcctcagttctc

SEQ ID NO: 43 - DNA, 60 bp, artificial
  PCR primer
gacgaattctctagaagatctctcgaggagctcaagcttctgtacagtgaccggtgactc

SEQ ID NO: 44 - DNA, 27 bp, artificial
  PCR primer
gacgaattccgatgaatgtgtgtcctg

SEQ ID NO: 45 - DNA, 79 bp, artificial
  PCR primer
gcacatatgatttaaatccctaatgttgaccctaatgttgaccctaatgttgagcggccgcgtttaaacgaattcgccc

SEQ ID NO: 46 - DNA, 71 bp, artificial
  PCR primer
cgtaagcttatttaaatccctaatgttgaccctaatgttgaccctaatgttgagaccggtgactctttctg

SEQ ID NO: 47 - DNA, 6576 bp, Aspergillus oryzae
gtctagaatcagttatgctgccccgtgaagtagcgtactcatcgggtgaggcatatgactctggtggtaatggcttttccatccagttttgatgtccttcgcaggacaagtctttggtcctcgcggagaacgagcgagaagcttgatggccacgagctgcagcagggttgttcttgtaggacaccgctcgagtctgcgggacatcgacagatccgtaagtctgatcgctatcagtttcgtaatccaatgcggtaggtttcatgtcgtgagactgctgccgtcggtgcgatggtgcgcgtggtacactattcagagggacggattggggaggtattgagcctccattagtaggcctctgggagctagctgtagccatccttcaaagctcccggattcttcacgacacacactacccagaaagcttccgtactttatcaccggtaaaaatcaaatgcgcggggaatagaaaggttagcgacaccaataaagatcgtaaaagaaatcaattcgtatccaaatttatgatagtagcagatgatttgaccagaccagttcgtgttagtacatggtgatggcactggaagaataaatcatcgcttaattcgcagagttgaaagttgcggagtagtatacaagcgatgtagtatatggattgtaacaagggtggatttgtacagtatgagaggaaggtaagatgatcgaataccgcgatttcaaccaggggggaaaagaatggaaaaaattaatgagatgacagaaaaatcaacaggctaacaggattatattggagtgacgaattaatctcattgtgctggctaatgagaattaagtcggtggctggcaatggcgaaccagcacagttccatgcggggctgactcgtcagtcatcctgcattaaacatgctcccgccaggcaccacgactggctcacgttcccctttttctaagaaaagaaattagctattagctttcgaaggagaggaattatcggactaggagcatgaaagggtacctggatagaaaacatgacgaaaagtatccatcgtgctgtgtcagtaatccgtgtataataataagtagtactgtagtcgtgttgcagcagctcaccgactggatgcatccgctggtgaatgagcaaatcatgaatgcaccgagtcagcattgagcatagtcggttttagtaacattgtaaagaagataatggcattcttgtttttgagacatgtttcccatccgctgtccatcatgggatgtttcatttagattctccaatcggcggtgtgatcagtaatcttatttcaacatctaccgtagattcaatgctgcctcttagagcaattagttacggttgtgaatgggccgagctgttgatataaaaagtaattccgaggggactgttgaggcattttgaaaattacggagcacatacatcgaagggggtattgtatggaccgcgatctagatctgcataccgacttcgatttcacaatgttgattcgtctattgtagtcgtcatctcattttattgtacatacagtaagaaagggaaaactcagaataagaaaagaagtcaccaaaacagggctgaatagtcctttcaactctttttacttttttttaggttcttagtaactgcctcccgtccatgttacttagtatacggagtacttttcatatcatcccttcacttgaagtcagaggggttttggctgtaaggagtagaggcttagctgctgataagtcaaggaaggtactctgtacagtgcttattagaattttgcaagaatgttcatctagtcgaaatctgagacgtggggccaaaatcttctagagtatgtactgtaattaattaatatttagcactgaatgaaaaggctacctgagcactacgatttaagtcagtggagaacaggttaaagttaaacaatgaatttgtcgcctaaggcaaaaggttccgctgtttccccgaagtgaacaccattctcgaactccgcctccgcaacaacttcaccgagcagtgctcattgaatacataaccatttcataccccccttgctgtttatattgcatatttcttgtctttatagtactcttttccttgaagttgctttctgaatcgcaagccatctactgaactgtctggtactgttcttattgatggcaactatcttatttgtcctcatctcgccttactactgataggagcctatctcgattcacgctactcccataatgcggctttccgaaagtgcaacggtagcgttcggccttttttgtgccgcgacggcatcagcccatcctcgacgctcctacgagacgcgcgatttctttgctcttcatcttgacgattccacctccccagacgagatcgcccaacgcctcggagcacgccacgagggtcaagtaggtgaactcacacaacaccataccttctctatacccaaggagaacggtgcagacctcgatgcgctgctcgaacatgcacgaatcagaaaaaggtcaagtcgtgccgaaggacgtggcatgacgttggacaaggaaagagatttgagtggtatactctggtctcaaaagttagccccaaaacagcgactagtaaagagggctcctccaacaaatgtggcgtcgagggggtctgtgaaagaagaggaccccgtagctgcccaagctcagaaacggattgcctcttcacttgggattacagatcccattttcggcggacagtggcatctttacaacactgtccaggttggccatgatctcaatgtttcggacgtctggttagaaggtatcaccgggaaaggtgtcatcacggctgtggtcgatgacggactggacatgtacagcaacgacctcaaaccgaactactttgctgagggctcctacgattttaacgaccatgtaccggagcccagaccgcggcttggtgatgatagacacggcacaagatgtgctggtgaaattggagcagctaggaatgatgtctgtggagtaggcgttgcatacgacagccaagttgccggaattcggattttgtccgcacccattgacgacgcagatgaggctgctgccatcaactatggctttcagcgcaatgatatatattcatgctcttggggccctccggatgatggcgccacgatggaggcgccagggattcttatcaaacgagctatggttaacggtatccaaaatggccgaggaggcaaaggttctattttcgtctttgcagctggaaatggtgcagggtacgatgacaactgcaatttcgacggctacacaaacagcatttacagcatcaccgtcggcgctattgatcgagagggcaaacatcccagctactcggaatcatgctctgcccagttggttgtcgcttatagcagtggctcgagtgacgcgattcataccactgacgttggaactgataaatgttattcacttcacggcggaacttctgccgcaggcccgctagctgcgggtactattgccctcgctcttagtgcccgaccggaactaacttggcgagatgcccagtacctgatgatagagaccgcagttcccgtccacgaagacgacgggagctggcagactaccaaaatggggaagaagtttagccatgactgggggtttgggaaagtagatgcatattcactggtacagctggccaagacgtgggagctggtgaaaccacaggcgtggttccactcaccgtggctgcgggtgaagcatgaaatcccacaaggtgaccagggtcttgccagctcatacgaaattaccaaggatatgatgtatcaggccaatatcgagaaactggaacatgtcactgtgaccatgaatgtaaatcacactcgccgaggcgacatcagtgtggagttgcgcagccccgaaggtatcgtcagtcatctgagtacagcgcggcggtcagataatgcaaaggctggctatgaagactggacgtttatgactgtggctcattggtatgtatttgctcccgtaatttagttttcgttgtcagtcctgacatttccattcaggggtgagtccggtgttggaaagtggacggtcattgtgaaggataccaatgtcaatgatcatgttggagaattcatcgactggcggctcaacctctggggactttcgatcgacggctccagccagccccttcatcctatgcccgatgagcatgacgatgaccactcgattgaagatgccattgttgttaccactagtgttgatcctctcccaactaagactgaagccccacctgtcccaactgatcccgtggatcgtcctgtgaacgcaaagccatctgcgcagccaacgacgccttcagaggctcctgctcaagagacatctgaagctcccaccccgacgaaacccagttctactgaatcaccttctaccaccacctctgcggatagctttttgccgtccttcttccccacgttcggcgcttcaaaacggacccaggcttggatttacgctgcgatcagttcgatcattgtattctgtattggccttggtgtctacttccacgtacagcgacgaaagcgtctgcgtaatgatccgcgtgatgactacgatttcgaaatgatagaagatgaggatgaaacgcaggctatgaacgggcgttcgggtcgtacacaacgccggggcggcgagctttacaatgctttcgctggggagagcgacgaagaacctttgtttagcgacgatgaggatgagccttatagggatcatgcccttagtgaagatcgggaacggcgagggagcacaagtggtgaccatgctcggtcatagtttggactaggctttgcatttgcttctaccctataatggtactccttcggcgcgttcccgctatatcagatgagatgtgttacatggatattgtgaattactgatgttgaacgaaggctgctgtatataattctgacttgattgacaaatagactcataaaggacatgcataggggtatcgtaaatagctgcaaagcgcgctacaagtaaaaaagtggatggggttgatagagttgctggataagccagtcttggcgcttgggccgatgacgctggtgcggcctcttctccaactgccgccattgactgctccactgctgcctccgcaacttctcaacctcccatttatcaatctaccaccagcaaccatagctcctcatatcccgaactacgctatatctggtctctccggtgatataaattgcgtgcgagtttcttttgacttgtacaattacctgtgtgaagttgccgcttccctaacggcaacccctcgatggatcagcacgatgactttgacaatgtctcatggagacacgaacctgagagcgacatctctcgtcctaccacttcgggtactgatgccgaagagtcgcctgcgactagtcacgatgccaatggcaagcggagaatgagctcagctcatgaaaacccacaagccgggccactggcagatgcggtcgatctagcaggtatcggggatggggtgttggaatgccgggtggattcgcctctgaaagaaaatgacggtactaaagacgcatacatctcttatttggttacaacacaagtgagttgccccgtttccccgggagttacctgcggcttgttcatggcagtccactgtactgacgggagagacatcgcagaccgatttcaagtctttccagaggtcagaatttgcagtgcgcagacgatttacggatttcttcttcctatacaagacactctatcgggaatatcctgcttgcgcggtcccacctctacccgacaagcataaaatggaatacgtacggggggatcggttcgggcccgaattcactacacggcgtgcgtggtcgctacatcgatttctaaagcgcttggcattacatccggtgcttcgccgggctcccttgcttgctatattcctagaatctcccgactggaatgcgcatatgcggcttcacacttctcgcacatccaccaatccgtcggacaacagcggtgcccccggaatttttgacaactttaccgataccttcgtaaatgcgtttacgaaagtccacaagcctgatcgccggttcattgaggttcgagagaaggcagataaattggatgaagatctcaatcacgtagagaaaatcgtcgctagggttgctcggcgtgagtccgatttagagaccgactataatgagctcgccacacaattccgcaagttggtgtctctggagccaaacgtcgaggttcccctacaggtattcgcggcgtcggtggaggagacgggacgtgggctcaaaggtctcaaagatcacacggatcaaaactaccttggctcgctccgggatatggaggcctacattctgtccctcaaggcgcttctaaaaacccgtgagcagaaacaactcgactttgaagccctagtggattaccgcaacaaagccgtgagcgagcgcgactcgctcgccaccaacccatcatcctactatgcctctaatcccctgacctcatcgcctgcgtccttcatccgctccaagatggaagatatgcgcggggtcgacctgcaggcatgcaagctta

SEQ ID NO: 48 - DNA, 1212 bp, Aspergillus oryzae
atgcagtccatcaagcgtaccttgctcctcctcggagctatccttcccgcggtcctcggtgcccctgtgcaggaaacccgccgggccgctgagaagcttcctggaaagtacattgtcacattcaagcccggcattgacgaggcaaagattcaggagcataccacctgggctaccaacattcaccagcgcagtctggagcgtcgtggcgccactggcggtgatcttcctgtcggtattgagcgcaactacaagatcaacaagttcgccgcctatgcaggctctttcgacgatgctaccattgaggagattcgcaagaacgaagatgttgcctacgtcgaggaggaccagatctactacctcgatggcctgactacccagaagagtgccccctggggtctgggcagcatttcccacaagggccagcagagcaccgactacatctacgacactagtgccggcgagggcacctatgcctacgtggtggatagcggtgtcaatgtcgaccatgaggagttcgagggccgcgccagcaaggcctacaacgctgccggtggtcagcatgtggacagcattggccatggcacccacgtttccggcaccattgctggcaagacttatggtatcgccaagaaggccagcatcctttcggtcaaagttttccagggtgaatcgagcagcacttccgtcattcttgacggcttcaactgggctgccaacgacattgttagcaagaagcgtaccagcaaggctgcaatcaacatgagcttgggcggtggctactctaaggctttcaacgatgcggtcgagaacgcattcgagcagggtgttctctcggttgtcgctgccggtaacgagaactctgatgccggccaaaccagccctgcctctgcccctgatgccatcactgttgccgctatccagaagagcaacaaccgcgccagtttctccaactttggcaaggtcgttgacgtcttcgctcccggtcaagatatcctttctgcctggattggctcttcctctgccaccaacaccatctctggtacctccatggctactccccacattgtcggcctgtccctctacctcgctgcccttgagaacctcgatggccccgctgccgtgaccaagcgcatcaaggagttggccaccaaggacgtcgtcaaggatgttaagggcagccctaacctgcttgcctacaacggtaacgcttaa

SEQ ID NO: 49 - DNA, 2071 bp, Aspergillus oryzae
atgcggggtcttctactagctggagcccttggcctacctttggccgtccttgcgcatccgacccatcatgcacatggacttcaacgtcgcacagttgacttgaactcattccgtttgcaccaggcagcgaagtatatcaatgcgactgagtcttcgagtgatgtttcatcttctttctctcccttcaccgagcaaagctacgtggagacggccactcagctcgtgaagaatatcctgccagatgctaccttccgtgtcgtcaaggatcattacattggtagcaatggggtcgctcatgtcaattttcgtcagacggtccatggccttgacattgacaatgcggacttcaatgtcaatgtacgctgcagtccacctatactatgttcggtgctaaccacttcatttaggttgggaaaaatggaaagatcttttcctatggccactcattttatacgggcaaaatccccgatgccaatcctttgacgaagcgggattataccgaccctgtagcggctctcagaggaaccaacgaagctttacagctttctatcactctagatcaagtgtctactgaggctaccgaggacaaagagtccttcaatttcaagggagtctctggcaccgtttcggatcccaaggctcagttggtctacttggtaaaggaagatggcagccttgctttgacctggaaggtggagacagatattgacagcaactggctgttgacctacatcgatgccaataccggcaaagatgtccatggtgtggttgactacgtagccgaggcagattaccaagtatagtgagtattttaagaatgtgacttggactgtagaatgaagctgacacaccaccacagtgcatggggtattaatgatcccacggagggccctcgcaccgtcatcagcgatccatgggattcgtccgcatctgcgttcacctggatcagtgacggagaaaacaactataccacaactcgcggcaacaacggtatcgcgcagtcgaaccctaccggtggatcgcagtacttgaagaactaccggcctgatagccccgatttgaaattccaatacccctattcgctcaacgccacacccccagagtcctatattgatgcgtctatcactcagcttttctacactgccaacacgtaccacgacctactctacactctgggcttcaacgaggaggccggtaatttccagtacgataacaatggaaaaggaggtgctggaaacgactacgtgatcctcaatgctcaggacggttctggcaccaataacgccaacttcgctacgcccccggatggacagcccggccgcatgcgcatgtacatttggaccgagtcccagccttaccgtgacggctccttcgaggctggtattgtgattcacgagtatactcacggtctctctaaccggctcactggaggacctgctaactctcgctgcttgaatgcccttgaatccggcggaatgggtgaaggttggggagacttcatggccacggcaattcggctcaaggccggcgatactcactcgaccgattataccatgggtgaatgggctgcaaacaagaaaggtggcatccgtgcttacccattctcaacctccctggaaaccaaccctctcacctacaccagtctcaatgaattggacgaagtgcatgccatcggcgcggtgtgggctaacgtattgtacgagctgttgtggaacttgatcgataagcacggcaagaatgacgggccaaagcccgagttcaaggatggagttccgactgacggcaagtatctcgccatgaagctggtgattgatggcatagcattgtaagtgccaacctcgtttcctctttctacctatcgcaggggctaaccttgacttttaggcaaccttgcaaccccaactgtgtccaggctcgcgacgccatcctcgatgccgataaggctctcaccgatggtgctaacaagtgcgagatttggaaggcgtttgctaagcgtggtttgggtgaaggcgctgaataccatgcgtctcgtcgggtgggcagtgataaggtgccctctgatgcttgctag

SEQ ID NO: 50 - DNA, 1488 bp, Aspergillus oryzae
atgagaggcatcctcggcctttccctgctgccactactagcagcggcctcccccgttgctgttgactccatccacaacggagcggctcccattctttcggcctcaaatgccaaagaggttccagactcttacattgtcgtcttcaagaagcatgtttccgctgaaacggctgctgctcatcacacctgggtgcaggacatccacgattcgatgactggacgcatcgacctgaagaagcgctctctttttggtttcagtgatgacctttacctcggtctcaagaacaccttcgatatcgccgggtccctagcgggctactccggacatttccatgaggatgtgatcgagcaggtccggagacatcctgatgttgaatacatcgagaaagacaccgaagtccacaccatggaggagacaaccgagaagaatgctccctggggcttggctcgtatctctcaccgtgacagcctctcgttcggtacctttaacaagtacctgtatgcttcggaaggcggtgagggtgtcgatgcttatactattgacactggtatcaacattgagcatgtcgatttcgaggatcgagcacactggggaaagaccatccctagcaatgatgaggatgcggatggcaacggacacggaactcactgctccggaaccattgctggtaagaagtacggtgttgccaagaaggccaacatctatgccgtcaaggtcttgaggtccagcggttctggcactatgtccgatgtcgttctgggtgtcgagtgggccgtccagtcccacctcaagaaggctaaggacgccaaagatgccaaggtcaagggtttcaagggcagcgttgccaacatgagtcttggtggtgccaagtccaggacccttgaggctgctgtcaatgctggtgttgaggctggtcttcacttcgccgttgctgctggtaacgacaatgccgatgcctgcaactactcccctgctgccgctgagaatgccatcactgtcggtgcctcgacccttcaggatgagcgtgcttacttctccaactacggaaagtgcactgacatctttgccccgggtcccaacattctttccacctggactggcagcaagcacgctgtcaacaccatctctggaacctctatggcttctcctcacattgctggtctgctggcctacttcgtttctctgcagcctgctcaggactctgctttcgctgtcgatgagcttactcctgccaagctcaagaaggatatcatctccatcgccacccagggtgcccttactgatatcccatctgacacccccaaccttctcgcctggaacggcggtggtgccgacaactacacccagattgtcgccaagggtggatacaaggccggcagtgacaaccttaaggaccgctttgacggactagtcaacaaggccgagaagttgctcgctgaggagcttggagctatttacagtgagatccagggtgctgttgttgcatag

SEQ ID NO: 51 - DNA, 2511 bp, Aspergillus oryzae
atgcggctttccgaaagtgcaacggtagcgttcggccttttttgtgccgcgacggcatcagcccatcctcgacgctcctacgagacgcgcgatttctttgctcttcatcttgacgattccacctccccagacgagatcgcccaacgcctcggagcacgccacgagggtcaagtaggtgaactcacacaacaccataccttctctatacccaaggagaacggtgcagacctcgatgcgctgctcgaacatgcacgaatcagaaaaaggtcaagtcgtgccgaaggacgtggcatgacgttggacaaggaaagagatttgagtggtatactctggtctcaaaagttagccccaaaacagcgactagtaaagagggctcctccaacaaatgtggcgtcgagggggtctgtgaaagaagaggaccccgtagctgcccaagctcagaaacggattgcctcttcacttgggattacagatcccattttcggcggacagtggcatctttacaacactgtccaggttggccatgatctcaatgtttcggacgtctggttagaaggtatcaccgggaaaggtgtcatcacggctgtggtcgatgacggactggacatgtacagcaacgacctcaaaccgaactactttgctgagggctcctacgattttaacgaccatgtaccggagcccagaccgcggcttggtgatgatagacacggcacaagatgtgctggtgaaattggagcagctaggaatgatgtctgtggagtaggcgttgcatacgacagccaagttgccggaattcggattttgtccgcacccattgacgacgcagatgaggctgctgccatcaactatggctttcagcgcaatgatatatattcatgctcttggggccctccggatgatggcgccacgatggaggcgccagggattcttatcaaacgagctatggttaacggtatccaaaatggccgaggaggcaaaggttctattttcgtctttgcagctggaaatggtgcagggtacgatgacaactgcaatttcgacggctacacaaacagcatttacagcatcaccgtcggcgctattgatcgagagggcaaacatcccagctactcggaatcatgctctgcccagttggttgtcgcttatagcagtggctcgagtgacgcgattcataccactgacgttggaactgataaatgttattcacttcacggcggaacttctgccgcaggcccgctagctgcgggtactattgccctcgctcttagtgcccgaccggaactaacttggcgagatgcccagtacctgatgatagagaccgcagttcccgtccacgaagacgacgggagctggcagactaccaaaatggggaagaagtttagccatgactgggggtttgggaaagtagatgcatattcactggtacagctggccaagacgtgggagctggtgaaaccacaggcgtggttccactcaccgtggctgcgggtgaagcatgaaatcccacaaggtgaccagggtcttgccagctcatacgaaattaccaaggatatgatgtatcaggccaatatcgagaaactggaacatgtcactgtgaccatgaatgtaaatcacactcgccgaggcgacatcagtgtggagttgcgcagccccgaaggtatcgtcagtcatctgagtacagcgcggcggtcagataatgcaaaggctggctatgaagactggacgtttatgactgtggctcattggggtgagtccggtgttggaaagtggacggtcattgtgaaggataccaatgtcaatgatcatgttggagaattcatcgactggcggctcaacctctggggactttcgatcgacggctccagccagccccttcatcctatgcccgatgagcatgacgatgaccactcgattgaagatgccattgttgttaccactagtgttgatcctctcccaactaagactgaagccccacctgtcccaactgatcccgtggatcgtcctgtgaacgcaaagccatctgcgcagccaacgacgccttcagaggctcctgctcaagagacatctgaagctcccaccccgacgaaacccagttctactgaatcaccttctaccaccacctctgcggatagctttttgccgtccttcttccccacgttcggcgcttcaaaacggacccaggcttggatttacgctgcgatcagttcgatcattgtattctgtattggccttggtgtctacttccacgtacagcgacgaaagcgtctgcgtaatgatccgcgtgatgactacgatttcgaaatgatagaagatgaggatgaaacgcaggctatgaacgggcgttcgggtcgtacacaacgccggggcggcgagctttacaatgctttcgctggggagagcgacgaagaacctttgtttagcgacgatgaggatgagccttatagggatcatgcccttagtgaagatcgggaacggcgagggagcacaagtggtgaccatgctcggtcatag

SEQ ID NO: 52 - protein, 403 aa, Aspergillus oryzae
MQSIKRTLLLLGAILPAVLGAPVQETRRAAEKLPGKYIVTFKPGIDEAKIQEHTTWATNIHQRSLERRGATGGDLPVGIERNYKINKFAAYAGSFDDATIEEIRKNEDVAYVEEDQIYYLDGLTTQKSAPWGLGSISHKGQQSTDYIYDTSAGEGTYAYVVDSGVNVDHEEFEGRASKAYNAAGGQHVDSIGHGTHVSGTIAGKTYGIAKKASILSVKVFQGESSSTSVILDGFNWAANDIVSKKRTSKAAINMSLGGGYSKAFNDAVENAFEQGVLSVVAAGNENSDAGQTSPASAPDAITVAAIQKSNNRASFSNFGKVVDVFAPGQDILSAWIGSSSATNTISGTSMATPHIVGLSLYLAALENLDGPAAVTKRIKELATKDVVKDVKGSPNLLAYNGNA

SEQ ID NO: 53 - protein, 634 aa, Aspergillus oryzae
MRGLLLAGALGLPLAVLAHPTHHAHGLQRRTVDLNSFRLHQAAKYINATESSSDVSSSFSPFTEQSYVETATQLVKNILPDATFRVVKDHYIGSNGVAHVNFRQTVHGLDIDNADFNVNVGKNGKIFSYGHSFYTGKIPDANPLTKRDYTDPVAALRGTNEALQLSITLDQVSTEATEDKESFNFKGVSGTVSDPKAQLVYLVKEDGSLALTWKVETDIDSNWLLTYIDANTGKDVHGVVDYVAEADYQVYAWGINDPTEGPRTVISDPWDSSASAFTWISDGENNYTTTRGNNGIAQSNPTGGSQYLKNYRPDSPDLKFQYPYSLNATPPESYIDASITQLFYTANTYHDLLYTLGFNEEAGNFQYDNNGKGGAGNDYVILNAQDGSGTNNANFATPPDGQPGRMRMYIWTESQPYRDGSFEAGIVIHEYTHGLSNRLTGGPANSRCLNALESGGMGEGWGDFMATAIRLKAGDTHSTDYTMGEWAANKKGGIRAYPFSTSLETNPLTYTSLNELDEVHAIGAVWANVLYELLWNLIDKHGKNDGPKPEFKDGVPTDGKYLAMKLVIDGIALQPCNPNCVQARDAILDADKALTDGANKCEIWKAFAKRGLGEGAEYHASRRVGSDKVPSDAC

SEQ ID NO: 54 - protein, 495 aa, Aspergillus oryzae
MRGILGLSLLPLLAAASPVAVDSIHNGAAPILSASNAKEVPDSYIVVFKKHVSAETAAAHHTWVQDIHDSMTGRIDLKKRSLFGFSDDLYLGLKNTFDIAGSLAGYSGHFHEDVIEQVRRHPDVEYIEKDTEVHTMEETTEKNAPWGLARISHRDSLSFGTFNKYLYASEGGEGVDAYTIDTGINIEHVDFEDRAHWGKTIPSNDEDADGNGHGTHCSGTIAGKKYGVAKKANIYAVKVLRSSGSGTMSDVVLGVEWAVQSHLKKAKDAKDAKVKGFKGSVANMSLGGAKSRTLEAAVNAGVEAGLHFAVAAGNDNADACNYSPAAAENAITVGASTLQDERAYFSNYGKCTDIFAPGPNILSTWTGSKHAVNTISGTSMASPHIAGLLAYFVSLQPAQDSAFAVDELTPAKLKKDIISIATQGALTDIPSDTPNLLAWNGGGADNYTQIVAKGGYKAGSDNLKDRFDGLVNKAEKLLAEELGAIYSEIQGAVVA

SEQ ID NO: 55 - protein, 836 aa, Aspergillus oryzae
MRLSESATVAFGLFCAATASAHPRRSYETRDFFALHLDDSTSPDEIAQRLGARHEGQVGELTQHHTFSIPKENGADLDALLEHARIRKRSSRAEGRGMTLDKERDLSGILWSQKLAPKQRLVKRAPPTNVASRGSVKEEDPVAAQAQKRIASSLGITDPIFGGQWHLYNTVQVGHDLNVSDVWLEGITGKGVITAVVDDGLDMYSNDLKPNYFAEGSYDFNDHVPEPRPRLGDDRHGTRCAGEIGAARNDVCGVGVAYDSQVAGIRILSAPIDDADEAAAINYGFQRNDIYSCSWGPPDDGATMEAPGILIKRAMVNGIQNGRGGKGSIFVFAAGNGAGYDDNCNFDGYTNSIYSITVGAIDREGKHPSYSESCSAQLVVAYSSGSSDAIHTTDVGTDKCYSLHGGTSAAGPLAAGTIALALSARPELTWRDAQYLMIETAVPVHEDDGSWQTTKMGKKFSHDWGFGKVDAYSLVQLAKTWELVKPQAWFHSPWLRVKHEIPQGDQGLASSYEITKDMMYQANIEKLEHVTVTMNVNHTRRGDISVELRSPEGIVSHLSTARRSDNAKAGYEDWTFMTVAHWGESGVGKWTVIVKDTNVNDHVGEFIDWRLNLWGLSIDGSSQPLHPMPDEHDDDHSIEDAIVVTTSVDPLPTKTEAPPVPTDPVDRPVNAKPSAQPTTPSEAPAQETSEAPTPTKPSSTESPSTTTSADSFLPSFFPTFGASKRTQAWIYAAISSIIVFCIGLGVYFHVQRRKRLRNDPRDDYDFEMIEDEDETQAMNGRSGRTQRRGGELYNAFAGESDEEPLFSDDEDEPYRDHALSEDRERRGSTSGDHARS

SEQ ID NO: 56 - protein, 7 aa, artificial
  N-terminal
DIQMTQS

SEQ ID NO: 57 - protein, 8 aa, artificial
  N-terminal
EGQLVQSG

SEQ ID NO: 58 - protein, 7 aa, artificial
  N-terminal
EIVLTQS

SEQ ID NO: 59 - protein, 8 aa, artificial
  N-terminal
EVQLLQSG

SEQ ID NO: 60 - DNA, 29 bp, artificial
  PCR primer
gacggatccaccatggcgcgactatcgag

SEQ ID NO: 61 - DNA, 20 bp, artificial
  PCR primer
gacgaattctgccatttggg

SEQ ID NO: 62 - DNA, 2694 bp, Aspergillus oryzae
ctgcagagcgatggccgtaccagccatgctgtccttttctctggcctcaagcattttaaaaaagctgatctcttcctctcacgtccttcgttcagcaacttctctcttttgcctcaactttcccttccttcccccttccattccgtcgcctgtggttgggttctctttttcttccttttgcttctctttcttgaatacaggaaactgtattgaagcacaagggatttactaccaacacgtctcccgataatcacacgcgtgccccttggcgaagaccaccactcgatatacacataggcacaatggcgcgactatcgagtcgcaacggtgcggccaagccgttcactgcttggactaccatcttctaccttctccttgttttcatcgcgcccctggcattcttcggtaccgcacacgctgaggaggactctgtccaagacaactatggaactgtaattggtattgatttgggaacaacctactcgtatgttctacagctgtgagacaagataaaggcgttgcaatgactaactccatcaacagttgtgttggtgtgatgcagaatggaaaggtcgagattctcgtcaacgaccaaggaaaccgtatcactccttcctacgtcgctttcaccgatgaggaacgcctggtcggtgacgccgctaagaaccaatacgccgccaaccccgtcaggaccatctttgacatcaagtgagtttctcgggttggttcgtatcatgaatcggtgtgctaatgtttcccaggcgtctgattggtcgcaagtacgatgacaaggatgtcaccaaggacaccaagaacttccccttcaaggttgtcaacaaggatggcaagcctgtcgtgaaggttgacgtgaacaagacccccaagaccttcactcctgaggaggtttccgccatggtcctcggaaagatgaaggagatcgctgagggctacctcggaaagtctgtcacccacgccgtcgtcaccgtccccgcctacttcaacgacgcccagagacaggctaccaaggacgctggtaccatcgccggtctgaacgttctccgtgttgtcaacgaacctaccgccgccgctatcgcctacggattggacaagactggtgatgagcgccaggtcatcgtctacgatcttggtggtggtactttcgatgtctcccttctctccattgacaacggtgtcttcgaggttttggctaccgctggtgacactcaccttggtggtgaggactttgaccaccgtgtcatggactacttcgtcaagcagtacaacaagaagaacaacgttgacatcaccaaggacctcaagtccatgggtaagctcaagcgcgaagtcgagaaggccaagcgtactctctcttcccagatgtctactcgcattgaaatcgagtctttccacaacggcgaggacttctctgagaccctcacccgtgctaagttcgaagagctgaacatggatctgttcaagaagaccctcaagcccgttgagcaggtgctcaaggacgccaaggttaagaagtccgaggttgacgacattgtccttgttggtggatctacccgtattcccaaggtccaggctcttctcgaggagttcttcggtggcaagaaggccagcaagggtattaaccctgatgaggctgttgctttcggtgctgccgtccagggtggtgtcctttccggtgaggccggtaccgaggatgtcgttctgatggacgtcaaccctcttaccctcggtatcgagaccactggcggtgtcatgactaagctcatcccccgtaacaccgttatccctactcgcaagtcccagatcttctccaccgccgctgataatcagcctaccgttcttatccaagtttgtaagtttaccttctggacgaaagccttctagcaacccacactaactgatcatagatgagggcgagcgttccttgaccaaggataacaacctcctcggcaagttcgagcttaccagcattccccccgctccccgtggtgttccccagattgaggtctccttcgatttggacgccaacggtatcctgaaggtcagcgccagcgacaagggcactggcaaggctgagtccatcaccatcaccaacgacaagggtcgtctctcccaggaggagatcgaccgcatggtcgctgaggccgaagagttcgctgaggaggacaaggccatcaagagcaagatcgaggcccgcaactctctggagaattatgccttcagcttgaagaaccaggtcaacgatgagaacggcttgggtggccagattgatgaggacgacaagcagaccatcctggacgccgtcaaggaggtcactgactggctcgaggacaacgctgccgaagccaccaccgaggacttcgaggagcagaaggagcagctgtccaacgtggcctaccccatcaccagcaagctctatggctctgctccggctgatgaggatgacgagccctctgggcatgacgaactgtaaaatatttagagggatgtatggagttttatcttgctttattggctggtttgaatcattttttatttgacctcgacacatgaggtggcaggaatagcatgataccttgaatattatcttactagcatcaatcacagcattctccatccccagttgtaaatctgtttcaacaggtagaaactaggctagtctcccaaatggcagaattc

SEQ ID NO: 63 - protein, 12 aa, artificial
  Alignment sequence 1
ACMSHTWGERNL

SEQ ID NO: 64 - protein, 14 aa, artificial
  Alignment sequence 2
HGWGEDANLAMNPS

(19) United States
(12) Patent Application Publication
(10) Pub. No.: US 2010/0062491 A1
(43) Pub. Date: Mar. 11, 2010

(54) Overexpression of the Chaperone BIP in a Heterokaryon

(75) Inventor: Jan Lehmbeck, Veksoe (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/519,480

(22) PCT Filed: Jan. 3, 2008
(86) PCT No.: PCT/EP08/50044

§ 371 Date: Jun. 16, 2009

Related U.S. Application Data
(60) Provisional application No. 60/883,574, filed on Jan. 5, 2007.

(30) Foreign Application Priority Data
Jan. 5, 2007 (DK) PA 2007 00015

(51) Int. Cl. C12P 21/00

(52) U.S. Cl. 435/71.1

(57) ABSTRACT

The present invention relates to a method for increasing the production yield of a secreted antibody or antibody fragment in a filamentous fungal host cell, comprising: recombinant expression of the antibody or antibody fragment and over-expressing a BiP chaperone protein.

### REFERENCE TO SEQUENCE LISTING

This application contains a Sequence Listing in computer readable form. The computer readable form is incorporated herein by reference.

### FIELD OF THE INVENTION

The present invention relates to a method of increasing the production yield of a secreted antibody in a filamentous fungal host cell.

### BACKGROUND OF THE INVENTION

Gene products which are highly overexpressed are often poorly secreted due to suboptimal folding. Correct folding and assembly of a polypeptide occurs in the ER and is a prerequisite for transport from the ER through the secretory pathway. The hsp70 family of chaperone proteins has previously been shown to be involved improving the secretion of over-expressed proteins (WO94/08012). In WO94/08012 is described a method for increasing secretion of an over-expressed gene product by effecting the expression of at least one hsp70 chaperone protein. Specifically two members of this family, KAR2 and BIP, are mentioned. The examples relate to expression of BIP, KAR2 or PDI and a heterologous polypeptide in yeast expression systems.

Shustra et al., 1998, (Nature Biotechnol. 16:773-777) describes increased levels of single-chain antibody fragments in S. cerevisiae by over-expression of BIP or PDI.

Kauffman et al., 2002, (Biotechnol. Prog. 18:942-950) also describes improved secretion of single chain antibody fragments by BIP over-expression in S. cerevisiae, however, BIP over-expression alone will not allow the cell to maintain high-level heterologous scFv expression.

In Smith et al., 2004, (Biotechnol. and Bioengineering 85:340-350) it is reported that increasing the level of BIP in S. cerevisiae led to a decrease in beta-glucosidase secretion.

BIP over-expression has also been studied in other expression systems than yeast. Punt et al., 1998, (Appl. Microbiol. Biotechnol. 50:447-454) describes the role of bipA in the secretion of homologous and heterologous proteins in Aspergillus. The effect of over-expression of BiPA in A. niger and A. awamori on heterologous protein expression levels is investigated. It is concluded that in Aspergillus increased BiPA levels do not result in improved levels of secreted heterologous proteins. It therefore seems that in e.g. yeast BiP overproduction may have a positive effect on the secretion of artificially produced proteins, whereas in certain mammalian cells it might have the opposite effect (Dorner et al., 1992, EMBO J. 11: 1563-1571) or no effect Punt et al., 1998 supra. Lombraña et al., 2004 (Appl. and Environmental Micrbiol. 70: 5145-5152) on the other hand report that for some proteins over-expression of bipA in A. awamori causes an increase in the secreted level of a heterologous protein, however, only up to a certain level of bipA overexpression. Some homologous proteins were not affected.

It therefore appears that only in bakers yeast have consistent results been obtained, and that in filamentous fungi such as Aspergillus sp. the effects are highly unpredictable.

### SUMMARY OF THE INVENTION

The invention provides a method for increasing the production yield of a secreted antibody or antibody fragment in a filamentous fungal host cell, comprising: recombinant expression of the antibody or antibody fragment and over-expression of a BiP chaperone protein.

### BRIEF DESCRIPTION OF DRAWINGS

FIG. 1 shows the results of three Western blots of light chain, heavy chain and light+heavy chain expression in a hybridoma cell (Hy) and an Aspergillus oryzae heterokaryon (As). The first gel shows expression of a light chain in a hybridoma cell and an A. oryzae heterokaryon (example 13), the second gel shows expression of a heavy chain in a hybridoma cell and an A. oryzae heterokaryon (example 13) and the third gel shows expression of both light and heavy chain in a hybridoma cell and an A. oryzae heterokaryon (example 13). The first lane of each gel is a standard protein marker used to evaluate the size of the proteins present in the Hy and As lane. The bands observed for the transformant (As) were identified as the heavy chain (50, 53 and 55 kD, probably different glycol forms) and the light chain (25 kD).

FIG. 2 shows the results of three Western blots of light chain, heavy chain and light+heavy chain expression by an A. oryzae heterokaryon (As). From left to right the first gel shows expression of a heavy chain by an A. oryzae heterokaryon (example 16), the second gel shows expression of both heavy chain an light chain by an A. oryzae heterokaryon (example 16) and the third gel shows expression of the light chain by an A. oryzae heterokaryon (example 16). The first lane of each gel is a standard protein marker used to evaluate the size of the proteins, the second lane is fermentation broth, the third shows the fermentation broth after purification with MepHyperCel and the fourth lane shows the fermentation broth after purification on a ProteinA column.

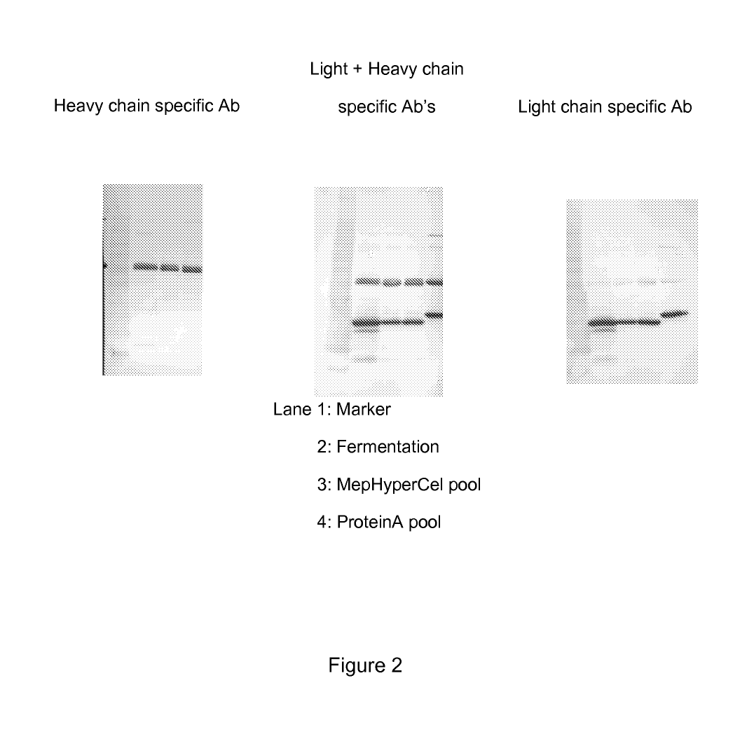

### DETAILED DESCRIPTION OF THE INVENTION

The present invention relates to a method for increasing the production yield of a secreted antibody or antibody fragment in a filamentous fungal host cell, comprising: recombinant expression of the antibody or antibody fragment and over-expression of a BiP chaperone protein.

### Monoclonal Antibody

In one particular embodiment of the present invention the antibody is a monoclonal antibody.

Physiologically antibodies are proteins produced by B-cells (plasma cells) on exposure to an antigen and which possess the ability to react in vitro and in vivo specifically and selectively with the antigenic determinants or epitopes eliciting their production or with an antigenic determinant closely related to the homologous antigen.

In its basic structure antibodies are comprised of two different polypeptide chains; a light chain (approximately 25 kDa) and a heavy chain (approximately 50-70 kDa). Each anti-body comprises of a total of four polypeptide chains; two light chains and two heavy chains. In any one antibody the two heavy and the two light chains are identical and the two heavy chains are linked to each other by disulfide bond(s) and each heavy chain is linked to a light chains by a disulfide bond, this gives the antibody its characteristic “Y” shape. The generic term “immunoglobulin” is used for all such proteins. Five different classes of heavy chains have been recognized, i.e. the mu, delta, gamma, alpha and epsilon chains which also defines the class of antibody, i.e. immunoglobulin M (IgM), immunoglobulin D (IgD), immunoglobulin G (IgG), immunoglobulin A (IgA) and immunoglobulin E (IgE), respectively. Furthermore, there are also sub-classes within these five main classes, e.g. in humans four different sub-classes of the gamma type have been recognized, i.e. gamma1, gamma2, gamma3 and gamma4 which produce IgG1, IgG2, IgG3 and IgG4. For the light chains two different types of chains have been recognized; the lambda and the kappa chains.

Both heavy and light chains are divided into distinct structural domains. A mu chain comprises from the N-terminal end a variable region (VH), a first, second, third and fourth constant region (CH1, 2, 3, 4), a delta chain comprises from the N-terminal end a variable region (VH), a first constant region (CH1), a hinge region, a second and a third constant region (CH2, 3), a gamma heavy chain comprises from the N-terminal end a variable region (VH), a first constant region (CH1), a hinge region, a second and third constant region (CH2, 3), an alpha chain comprises from the N-terminal end a variable region (VH), a hinge region, a second and third constant region (CH2, 3) and an epsilon chain comprises from the N-terminal end a variable region (VH), a first, second, third and fourth constant region (CH1, 2, 3, 4).

A light chain comprises a variable region (VL) and a constant region (CL). The different classes of heavy chains differ mainly in the number of constant regions, the presence or absence of a hinge region and the type and/or amount of glycosylation. However, all the different classes of heavy chains comprise a variable region, which is the region capable of binding to/recognizing the antigen.

Generally antibodies are divided into two groups; polyclonal and monoclonal antibodies. Polyclonal antibodies are different (with regard to class and/or subclass of the heavy and/or light chain and/or with regard to the antigenic determinant binding sequences) anti-bodies which bind to the same antigen. Monoclonal antibodies are identical (with regard to class and subclass of the heavy and light chain, and with regard to the antigenic determinant binding sequences) antibodies. In this context the terms “different” and “identical” refers to the amino acid sequence. Physiologically a monoclonal antibody is synthesized by a single clone of B lymphocytes or plasma cells. The identical copies of the antibody molecules produced contain only one class of heavy chain and one type of light chain. To obtain a homogenous population of antibodies methods for production of monoclonal antibodies have been developed. For example, Kohler and Millstein developed in the mid-1970s B lymphocyte hybridomas by fusing an antibody-producing B lymphocyte with a mutant myeloma cell that was not secreting antibody. Alternatively, antibodies (as Fab fragments or single chains) can be produced and improved by using display systems, e.g. phage display (Rodi, D. et al, 2002. Quantitative assessment of peptide sequence diversity in M13 combinatorial peptide phage display libraries. J Mol Biol 322, 1039-1052).

Different truncated forms of antibodies exist which previously was mainly generated by protease digestion but which today may also be generated by recombinant DNA technology. For example the protease papain cleaves an IgG molecule at the N-terminal side of the disulfide bonds in the hinge region to generate three fragments; two Fab fragments (the arms of the antibody) which each consist of the variable and first constant region of the heavy chain bound to the light chain by a disulfide bond and a Fc fragment which consist of the second and third constant region of both of the heavy chains bound to each other by disulfide bonds at the hinge region.

Another protease pepsin cleaves an IgG molecule at the C-terminal side of the disulfide bonds in the hinge region to generate a F(ab′)2 fragment and a number of small pieces of the Fc fragment. The F(ab′)2 fragment consist of the two Fab fragments from one molecute bound together by disulfide bonds at the hinge region.

In one particular embodiment the antibody or antibody fragment is selected from the group consisting of IgG1, IgG2, IgG3, IgG4, IgM, IgA, IgD, IgE, F(ab′)2, and Fab. In another particular embodiment the antibody is an IgG antibody.

### Chaperone Protein BiP

Immunoglobulin heavy chain binding protein (BiP) is a member of the Hsp70 chaperone family and is located in the Endoplasmic reticulum (ER). BiP has a weak ATPase activity. Binding of ATP is necessary for the release of peptides bound to BiP. BiP has a binding site selective for linear sequences of seven amino acids containing hydrophobic residues. BiP plays an important role in the protein traffic process by binding to the newly synthesized polypeptides and promoting their proper folding. It also binds to aberrant proteins, preventing them from leaving the endoplasmic reticulum to continue through the secretory pathway. The BiP protein appears to have a quality control function, discriminating between properly folded proteins to be exported and inadequately folded proteins. In one embodiment according to the invention BiP is BiP A from Aspergillus. In another embodiment BiP is BiP A from Aspergillus oryzae (Kasuya, T. Nakajima, H. Kitamoto, K.; “Cloning and characterization of the bipA gene encoding ER chaperone BiP from Aspergillus oryzae.”; J. Biosci. Bioeng. 88:472-478 (1999)). In another embodiment BiP is BiP A from Aspergillus awamori (Hijarrubia et al., 1997, Curr. Genet. 32:139-146).

According to the invention the endogenous copy of the Bip gene in the host cell could be controlled by its normal promoter, while one or more recombinant copies of an additional Bip gene is introduced into the host cell and expressed. Alternatively the endogenous copy of Bip could be inactivated or expression reduced. In one embodiment of the invention the Bip gene is present in the host cell in more than one copy. The expression level of Bip may further be increased by expressing a Bip gene from a strong promoter. In one embodiment the Bip gene expression is controlled by a promoter selected from the group consisting of A. oryzae TAKA amylase, NA2, NA2-tpi, glaA, tpi, gpd, tef1, pgk promoters.

### Protease Deficient Fungal Host Cells

According to the invention the level of a secreted protein product, such as an antibody product can be significantly improved by increasing the expression level of Bip A compared to an otherwise identical parent fungal host cell. The expression level of the antibody product may be further improved by reducing or eliminating the expression level of particular protease activities in the host cell. In one embodiment the protease activity to be reduced or eliminated according to the invention is selected from the group consisting of Alp, NpI, PepC and kexB.

### Serine Protease

In the context of this invention the protease activity to be reduced or eliminated is a serine protease with a broad range of activity between pH 4.5 and 11 which are released from a cell wall fraction. Analyses of the amino acid sequence of the serine proteases indicate homology to the subtilase subgroup of subtilisin-like serine proteases. As summarised by Siezen, et al. (1991. Protein Eng. 4:719-737) more than 50 subtilases have been identified from a wide variety of organisms, ranging from various species of bacteria, including gram positive and gram negative species, to fungi and yeast to higher eukaryotes, including worms, insects, plants and mammals. The amino acid sequences have been determined in more than 40 of these subtilases, and reveal that the mature region of the enzyme ranges from 268 to 1775 amino acids in length and a pre-pro-region of 27 to 280 amino acids in the N-terminal vicinity. In fungi and yeast, the variation is apparently smaller, with corresponding ranges of 279 to 397 and 105 to 121 in fungi, and 297 to 677 and 126 to 280 in yeast. Genomic clones of the entire coding region of the serine protease from Aspergillus oryzae, Aspergillus fumigatus and Aspergillus niger has been cloned (WO97/22705, Reichard et al. 2000. Int. J. Med. Microbiol. 290, 549-558, and Frederick et al. 1993. Gene 125. 57-64.). The primary structure was shown to share 29% to 78% homology with other sequenced subtilisins, and the three residues in the active site, Asp32, His 64 and Ser221 in subtilisin BPN′, were conserved.

In a particular embodiment, the serine protease of the subtilisin type is an Aspergillus oryzae serine protease (pepC), preferably encoded by a cDNA sequence comprising the nucleotide sequence presented as SEQ. ID. NO: 50 or a sequence homologous thereto. Particularly the homologous sequence has a degree of identity to SEQ ID NO: 50 of at least 60%, preferably at least 65%, more preferably at least 70%, more preferably at least 75%, more preferably at least 80%, more preferably at least 85%, even more preferably at least 90%, most preferably at least 95%, and even most preferably at least 97%.

Preferably the homologous sequence encodes a protease having an amino acid sequence which has a degree of identity to the amino acids of SEQ ID NO: 54 (i.e., the complete polypeptide including signal peptide and propeptide) of at least 60%, preferably at least 65%, more preferably at least 70%, more preferably at least 75%, more preferably at least 80%, more preferably at least 85%, even more preferably at least 90%, most preferably at least 95%, and even most preferably at least 97%, which have serine protease activity (hereinafter “homologous polypeptides”).

In a particular embodiment the filamentous fungal host cell in addition to or alternatively has reduced or eliminated expression of a serine protease of the kexin subfamily.

### Serine Protease of the Kexin Subfamily

Kexin is a Ca²⁺-dependent transmembrane serine protease that cleaves the secretory proproteins on the carboxyl side of Lys-Arg and Arg-Arg in a late Golgi compartment (Fuller and Thorner, 1989, PNAS 86:1434-1438; Mizuno et al., 1988, Biochem. Biophys. Res. Commun. 156:246-254). All members of the kexin subfamily are calcium-dependent, neutral serine proteases that are activated by the removal of the amino-terminal propeptide at a kexin-specific (auto) processing site. The active proteases all contain two additional domains, a subtilisin-like domain containing the catalytic triad and a conserved P or Homo B domain of approximately 150 residues. The P domain, which is absent in other subtilases, is essential for the catalytic activity and the stability of the protein. Aspergillus kexins are found in Aspergillus nidulans (Kwon et al., 2001, Mol. Cell. 12:142-147), A. niger (Jalving et al., 2000, Appl. Environ. Microbiol. 66:363-368), and A. oryzae (Mizutani et al., 2004, Eukaryotic Cell 3:1036-1048).

In a particular embodiment, the serine protease of the kexin subfamily is an Aspergillus oryzae serine protease (kexB), preferably encoded by a cDNA sequence comprising the nucleotide sequence presented as SEQ ID NO: 51 or a sequence homologous thereto.

Particularly the homologous sequence has a degree of identity to SEQ ID NO: 51 of at least 60%, preferably at least 65%, more preferably at least 70%, more preferably at least 75%, more preferably at least 80%, more preferably at least 85%, even more preferably at least 90%, most preferably at least 95%, and even most preferably at least 97%.

Preferably the homologous sequence encodes a protease having an amino acid sequence which has a degree of identity to the amino acids of SEQ ID NO: 55 (i.e., the complete polypeptide including signal peptide and propeptide) of at least 60%, preferably at least 65%, more preferably at least 70%, more preferably at least 75%, more preferably at least 80%, more preferably at least 85%, even more preferably at least 90%, most preferably at least 95%, and even most preferably at least 97%, which have serine protease activity (hereinafter “homologous polypeptides”).

In a particular embodiment the filamentous fungal cell according to the invention has the phenotype alp⁻, np1⁻, pepC⁻, and kexB⁻, wherein the alp gene is encoded by a nucleotide sequence which has at least 70% identity to SEQ ID NO: 48, the np1 gene is encoded by a nucleotide sequence which has at least 70% identity to SEQ ID NO: 49, the pepC gene is encoded by a nucleotide sequence which has at least 70% identity to SEQ ID NO: 50, and the kexB gene is encoded by a nucleotide sequence which has at least 70% identity to SEQ ID NO: 51.

In the above the nucleotide sequences referred to are the cDNA sequences (CDS without introns) corresponding to the mature mRNA after splicing.

### Heterokaryon

In a further embodiment according to the invention the fungal host cell is a heterokaryon fungal cell.

In the context of the present invention a “heterokaryon” is to be understood as a cell with at least two genetically different nuclei. Heterokaryons derive from fusion of two or more genetically different cells wherein the nuclei of said cells do not fuse resulting in a cell comprising two or more nuclei.

The heterokaryon fungus may be formed naturally between two or more fungi or it may be made artificially. When two or more genetically different fungi fuse the nucleus of each of the individual cells come to coexist in a common cytoplasm. One method to select for heterokaryons is to fuse two or more genetically different cells which each comprise a genome with a characteristic which renders the survival of each cell dependent on presence of the nucleus from the other cell. For example if two genetically different cells which each depends on a particular nutrient for survival and at the same time is independent of the nutrient the other cell depends on for survival is cultured in the a medium lacking both of the nutrients this will make only cells which arise as a fusion between each of the genetically different cells able to survive in this medium.

The heterokaryon filamentous fungus of the present invention may in particular contain nuclei from cells that are homozygous for all heterokaryon compatibility alleles. At least ten chromosomal loci have been identified for heterokaryon incompatibility: het-c, het-d, hete, het-i, het-5, het-6, het-7, het-8, het-9 and het-10, and more probably exist (see e.g. Perkins et al., “Chromosomal Loci of Neurospora crassa”, Microbiological Reviews (1982) 46: 462-570, at 478).

Formation of the heterokaryon filamentous fungus may in particular be performed by hyphal or protoplast fusion.

In particular the heterokaryon filamentous fungus of the present invention may be made by fusion of hyphae from two different strains of filamentous fungi, wherein the first nuclei of one of the strains contains a genome that results in a characteristic which renders the fungus dependent on the presence of the second nucleus from the other fungus for survival under the conditions provided for fusion to form the heterokaryon, and vice versa. Thus the nucleus of each strain of filamentous fungus confers a characteristic which would result in the failure of the fungus in which it is contained to survive under the culture conditions unless the nucleus from the other filamentous fungus is also present. Examples of characteristics which may be used to render the strains of filamentous fungi dependent on each other include, but are not limited to, a nutritional requirement, resistance to toxic compounds and resistance to extreme environmental conditions. For example if a first strain which requires the presence of a particular nutrient is cultured on a medium lacking said nutrient along with a second strain which does not require said nutrient for survival, the nucleus of the second strain will confer the ability of a fusion of the two strains to survive even in the absence of the particular nutrient. Furthermore, if the second strain similarly requires the presence of a particular nutrient different from the nutrient required by the first strain, then only fusions comprising a nucleus from each strain will survive in a medium lacking both of said nutrients.

Methods for formation of a heterokaryon filamentous fungus are described in U.S. Pat. No. 6,543,745.

Examples of filamentous fungi which may be fused to form a heterokaryon filamentous fungus include A. oryzae. In principle, more than two different strains of filamentous fungi may used to form a heterokaryon, such as 3 or 4 or 5 or 6 or 7 or 8 or 9 or 10 different strains. In particular the heterokaryon filamentous fungus of the present invention is formed by fusion of two different strains of filamentous fungi.

Examples of characteristics which make each of the strains of fungi (that are fused to form a heterokaryon filamentous fungus) dependent on the presence of the nucleus from the other fungus for survival under the conditions provided for the fusion include the selectable markers described above. In particular said characteristic may be a characteristic that makes the fungus autotroph. The culture media used for fusion of the different strains of fungi to form a heterokaryon filamentous fungus may be any media which does not complement the particular characteristic of the fungi. Examples of such media are well known to a person skilled in the art as they are generally used to select for recombinant fungi. In the case of fusion of different fungi, however, at least two different characteristics/markers are used for the selection. Examples of characteristics or markers which may be used include those described above as selectable markers useful for the nucleic acid construct. Examples of genes which may make a fungus autotroph include, but are not limited to: pyrG, hemA, niaD, tpi, facC, gala, biA, lysB, sC, methG and phenA. Thus if a fungus is negative for at least one of these genes said gene may be used as a selectable marker.

Methods of transformation of fungi are well known and may be performed as described below for the fungal host cells. Conditions for culturing a heterokaryon fungus are similar to those for culturing the fungi that it is derived from with the exception that the heterokaryon is cultured in a medium selecting for at least two different characteristics. The selection for at least two different characteristics needs at least to be maintained during formation of the heterokaryon but usually it also an advantage to keep this selection pressure, i.e. the selection for at least two characteristic during subsequent culturing to ensure the stability of the heterokaryon. Methods for culturing fungi are well known to a person skilled in the art.

### Genetic Modifications of the Host Cell

The host cell of the invention, in order to express significantly reduced levels of serine protease activity, and optionally serine protease of the kexin subfamily is genetically modified which may be achieved by using standard recombinant DNA technology known to the person skilled in the art. The gene sequences respectively responsible for production of the protease activity may be inactivated or partially or entirely eliminated. Thus, a host cell of the invention expresses reduced or undetectable levels of serine protease or expresses functionally inactive proteases.

In a particular embodiment, the said inactivation is obtained by modification of the respective structural or regulatory regions encoded within the protease genes of interest.

Known and useful techniques include, but are not limited to, specific or random mutagenesis, PCR generated mutagenesis, site specific DNA deletion, insertion and/or substitution, gene disruption or gene replacement, anti-sense techniques, or a combination thereof.

Mutagenesis may be performed using a suitable physical or chemical mutagenising agent. Examples of a physical or chemical mutagenising agent suitable for the present purpose include, but are not limited to, ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulfite, formic acid, and nucleotide analogues. When such agents are used, the mutagenesis is typically performed by incubating the cell to be mutagenised in the presence of the mutagenising agent of choice under suitable conditions, and selecting for cells showing a significantly reduced production of the protease of choice.

Measurements of the extracellular proteases activity of the alkaline and neutral metalloprotease I can be done as described by Markaryan et al. (1996) J Bacteriology 178, no 8, 2211-2215. In short the strain is grown in a media which induce the production of extracellular proteases. The broth is then separate from the mycelium and are assay for alkaline and metalloproteinase activity with the substrate Suc-Ala-Ala-Pro-Leu-pNa and Abz-Ala-Ala-Phe-Phe-pNa, respectively.

Measurement of the intracellular Kexin is done as described by Jalving et al. (2000) Applied and Environmental Microbiology 66, no 1, 363-368. In short strains are grown minimal media (COVE (1966) Biochim. Biophys. Acta 113, 51-56), mycelium is harvested, grounded and the membrane protein fraction were suspended in HEPES buffer and stored at −20° C. until used. The isolated membrane protein fraction were analysed for kexin activity by using the substrate Boc-Leu-Lys-Arg-MCA.

Measurement of pepC which are cell wall bound was done according to Reihard et al. (2000) Int. J. Med. Microbiol. 290, 85-96. In short strains are grown in minimal media (COVE (1966) Biochim. Biophys. Acta 113, 51-56), mycelium is harvested, grounded, and cell wall fragments were suspended in Na-citrate buffer and stored at −20° C. until used for proteinase assays. The isolated cell wall were analyse for pepC activity by an azocasein assay (Scharmann and Balke (1974) Physiol. Chem. 355, 443-450.

Modification may also be accomplished by the introduction, substitution or removal of one or more nucleotides in the structural sequence or a regulatory element required for the transcription or translation of the structural sequence. For example, nucleotides may be inserted or removed so as to result in the introduction of a stop codon, the removal of the start codon or a change of the open reading frame of the structural sequence. The modification or inactivation of the structural sequence or a regulatory element thereof may be accomplished by site-directed mutagenesis or PCR generated mutagenesis in accordance with methods known in the art. Although, in principle, the modification may be performed in vivo, i.e. directly on the cell expressing the metalloprotease, alkaline protease, and serine protease genes, it is presently preferred that the modification be performed in vitro as exemplified below.

A convenient way to inactivate or reduce the said protease production in a host cell of choice is based on techniques of gene interruption. In this method a DNA sequence corresponding to the endogenous gene or gene fragment of interest is mutagenised in vitro. Said DNA sequence thus encodes a defective gene which is then transformed into the host cell. By homologous recombination, the defective gene replaces the endogenous gene or gene fragment. It may be desirable that the defective gene or gene fragment also encodes a marker which may be used to select for transformants in which the respective genes encoding metalloprotease and/or alkaline protease have been modified or destroyed.

Methods for deleting or disrupting a targeted gene are specifically described by Miller, et al. (1985. Mol. Cell. Biol. 5:1714-1721); WO 90/00192; May, G. (1992. Applied Molecular Genetics of Filamentous Fungi. J. R. Kinghorn and G. Turner, eds., Blackie Academic and Professional, pp. 1-25); and Turner, G. (1994. Vectors for Genetic Manipulation. S. D. Martinelli and J. R. Kinghorn, eds., Elsevier, pp. 641-665).

Alternatively, the modification or inactivation of the DNA sequence may be performed by established anti-sense techniques using a nucleotide sequence complementary to a coding sequence for a metalloprotease, e.g. the nucleotide sequences presented as SEQ ID NO: 27, an alkaline protease encoding sequence, e.g. the nucleotide sequence shown in SEQ. ID. NO: 26, or a serine protease of the subtilisin type and optionally also of the kexin subfamily, e.g. the nucleotide sequences shown in SEQ ID NO: 28 and SEQ ID NO: 29. The anti-sense technology and its application are described in detail in U.S. Pat. No. 5,190,931 (University of New York).

Therefore, due to genetic modification, the host cell of the invention expresses significantly reduced levels of metalloprotease, alkaline protease, and serine protease of the subtilisin type activity. In a particular embodiment the host cell in addition expresses significantly reduced levels of a serine protease of the kexin subfamiliy. In a particular embodiment, the level of these proteolytic activities expressed by the host cell is individually reduced more than about 50%, preferably more than about 85%, more preferably more than about 90%, and most preferably more than about 95%. In another particular embodiment, these proteolytic activities in the host cell of the invention may be reduced in any combination. In a most particular embodiment, the product expressed by the host cell is essentially free from proteolytic activity due to any of the above proteases.

### Methods of Producing Proteins

One aspect of the invention provides a method for increasing the production yield of a secreted antibody or antibody fragment in a host cell of the invention, which the method comprises introducing into said host cell a nucleic acid sequence encoding the antibody product of interest, cultivating the host cell in a suitable growth medium thereby expressing and secreting the antibody product and expressing the BiP chaperone at increased levels compared to an otherwise identical parent host cell, followed by recovery of the secreted antibody product. The increase in the yield of secreted antibody in the host cell over-expressing BiP compared to an equivalent host cell which does not over-express BiP is particularly at least a factor 1.5, more particularly at least a factor 2, more particularly at least a factor 3, more particularly at least a factor 4, more particularly at least a factor 5, even more particularly at least a factor 6.

By one embodiment of the invention, the proteolytic activities of certain proteases: serine protease of the subtilisin type and/or serine protease of the kexin subfamily are additionally significantly reduced, thereby further improving the stability and increasing the yield of susceptible protein products synthesised by the host cell of the invention. More specifically, by the method of the invention, the host cell is genetically modified within structural and/or regulatory regions encoding or controlling the serine protease of the subtilisin type and/or serine protease of the kexin subfamily protease genes.

Thus, the host cell of the invention must contain structural and regulatory genetic regions necessary for the expression of the desired antibody product. The nature of such structural and regulatory regions greatly depends on the product and the host cell in question. The genetic design of the host cell of the invention may be accomplished by the person skilled in the art using standard recombinant DNA technology for the transformation or transfection of a host cell (vide, e.g., Sambrook et al., inter alia).

Preferably, the host cell is modified by methods known in the art for the introduction of an appropriate cloning vehicle, i.e. a plasmid or a vector, comprising a DNA fragment encoding the desired protein product. The cloning vehicle may be introduced into the host cell either as an autonomously replicating plasmid or integrated into the chromosome. Preferably, the cloning vehicle comprises one or more structural regions operably linked to one or more appropriate regulatory regions.

The structural regions are regions of nucleotide sequences encoding the desired protein product. The regulatory regions include promoter regions comprising transcription and translation control sequences, terminator regions comprising stop signals, and polyadenylation regions. The promoter, i.e. a nucleotide sequence exhibiting a transcriptional activity in the host cell of choice, may be one derived from a gene encoding an extracellular or an intracellular protein, preferably an enzyme, such as an amylase, a glucoamylase, a protease, a lipase, a cellulase, a xylanase, an oxidoreductase, a pectinase, a cutinase, or a glycolytic enzyme.

Examples of suitable promoters for heterologous protein expression in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Dada (WO 00/56900), Fusarium venenatum Quinn (WO 00/56900), Fusarium oxysporum trypsin-like protease (WO 96/00787), Trichoderma reesei beta-glucosidase, Trichoderma reesei cellobiohydrolase I, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

The cloning vehicle may also include a selectable marker, such as a gene product which complements a defect in the host cell, or one which confers antibiotic resistance. Examples of antibiotics useful as Aspergillus selection markers include hygromycin, phleomycin and basta. Other examples of Aspergillus selection markers include amdS, which encodes an enzyme involved in acetamide utilisation; pyrG, which encodes an enzyme involved in uridine biosynthesis; argB, which encodes an enzyme involved in arginine biosynthesis; niaD, which encodes an enzyme involved in the nitrate assimilation pathway; and sC, which encodes an enzyme involved in the sulfate assimilation pathway. Preferred for use in an Aspergillus host cell are the amdS and pyrG markers of Aspergillus nidulans or Aspergillus oryzae. Furthermore, selection may be accomplished by co-transformation, wherein the transformation is carried out with a mixture of two vectors and the selection is made for one vector only.

The procedures used to ligate the DNA construct of the invention, the promoter, terminator and other elements, respectively, and to insert them into suitable cloning vehicles containing the information necessary for replication, are well known to persons skilled in the art (vide e.g., Sambrook et al., 1989; inter alia).

The culture broth or medium used may be any conventional medium suitable for culturing the host cell of the invention, and formulated according to the principles of the prior art. The medium preferably contains carbon and nitrogen sources as well as other inorganic salts. Suitable media, e.g. minimal or complex media, are available from commercial suppliers, or may be prepared according to published recipes, as in: The Catalogue of Strains, published by The American Type Culture Collection. Rockville Md., USA. 1970.

The appropriate pH for fermentation will often be dependent on such factors as the nature of the host cell to be used, the composition of the growth medium, the stability of the polypeptide of interest, and the like. Consequently, although the host cell of the invention may be cultured using any fermentation process performed at any pH, it is advantageous that the pH of the fermentation process is such that acidic and/or neutral protease activities of the host cell are essentially eliminated or at least significantly reduced. Thus, removal of aspartic protease activity as described in WO 90/00192 may also be accomplished by raising the fermentation pH, and does not present any additional advantageous effect on the yield of a desired protein from host cells cultivated in the alkaline pH range.

If the pH of the fermentation process is within the range from 5 to 11, such as from 6 to 10.5, 7 to 10, or 8 to 9.5, the activity of acidic proteases, such as aspartic and serine proteases, and neutral proteases in the pH ranges above 7, will be reduced or blocked. Examples of enzymes produced under alkaline fermentation conditions include endoglucanases, phytases and protein disulfide isomerases.

However, the alkaline pH range will support alkaline protease activity in an unmodified host cell, which, in turn, may potentially result in degradation of the polypeptide product of interest. Consequently, in such cases the inactivation of the gene encoding alkaline protease is especially advantageous.

Inactivation of the alkaline protease gene of the invention is also especially advantageous for certain host cells, as the levels of acidic, neutral and alkaline protease activities vary from species to species. For example, the level of alkaline protease activity in the Aspergillus oryzae is higher than in Aspergillus niger.

After cultivation, the desired protein is recovered by conventional methods of protein isolation and purification from a culture broth. Well established purification procedures include separating the cells from the medium by centrifugation or filtration, precipitating proteinaceous components of the medium by means of a salt such as ammonium sulphate, and chromatographic methods such as ion exchange chromatography, gel filtration chromatography, affinity chromatography, and the like.

### Host Cells

The host cell of the invention is a filamentous fungus. It is advantageous to use a host cell of the invention in recombinant production of a polypeptide of interest. The cell may be transformed with the DNA construct encoding the polypeptide of interest, conveniently by integrating the DNA construct in one or more copies into the host chromosome. This integration is generally considered to be an advantage as the DNA sequence is more likely to be stably maintained in the cell. Integration of the DNA construct into the host chromosome may be performed according to conventional methods, e.g., by homologous or heterologous recombination. Alternatively, the cell may be transformed with an expression vector as described in the examples below in connection with the different types of host cells.

### Filamentous Fungal Host Cells

The host cell of the invention is a filamentous fungus represented by one of the following groups of Ascomycota, include, e.g., Neurospora, Eupenicillium (=Penicillium), Emericella (=Aspergillus), Eurotium (=Aspergillus).

In a preferred embodiment, the filamentous fungus belongs to one of the filamentous forms of the subdivision Eumycota and Oomycota as defined by Hawksworth et al., In, Ainsworth and Bisby's Dictionary of The Fungi, 8th edition, 1995, CAB International, University Press, Cambridge, UK. The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic.

In a more particular embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more particular embodiment, the filamentous fungal host cell is an Aspergillus cell. In another even more particular embodiment, the filamentous fungal host cell is an Acremonium cell. In another even more particular embodiment, the filamentous fungal host cell is a Fusarium cell. In another even more particular embodiment, the filamentous fungal host cell is a Humicola cell. In another even more particular embodiment, the filamentous fungal host cell is a Mucor cell. In another even more particular embodiment, the filamentous fungal host cell is a Myceliophthora cell. In another even more particular embodiment, the filamentous fungal host cell is a Neurospora cell. In another even more particular embodiment, the filamentous fungal host cell is a Penicillium cell. In another even more particular embodiment, the filamentous fungal host cell is a Thielavia cell. In another even more particular embodiment, the filamentous fungal host cell is a Tolypocladium cell. In another even more particular embodiment, the filamentous fungal host cell is a Trichoderma cell. In a most particular embodiment, the filamentous fungal host cell is an Aspergillus awamori, Aspergillus foetidus, Aspergillus japonicus, Aspergillus aculeatus, Aspergillus niger, Aspergillus nidulans or Aspergillus oryzae cell. In another particular embodiment, the filamentous fungal host cell is a Fusarium cell of the section Discolor (also known as the section Fusarium). For example, the filamentous fungal host cell may be a Fusarium bactridioides, Fusarium cerealis, Fusarium crookwellense, Fusarium culmorum, Fusarium graminearum (in the perfect state named Gibberella zeae, previously Sphaeria, synonym with Gibberella roseum and Gibberella roseum f.sp. ceralis), Fusarium graminum, Fusarium heterosporum, Fusarium negundi, Fusarium reticulatum, Fusarium roseum, Fusarium sambucinum, Fusarium sarcochroum, Fusarium sulphureum, Fusarium trichothecioides or Fusarium venenatum cell. In another preferred embodiment, the filamentous fungal host cell is a Fusarium strain of the section Elegans, e.g., Fusarium oxysporum. In another most particular embodiment, the filamentous fungal host cell is a Humicola insolens or Humicola lanuginosa cell. In another most particular embodiment, the filamentous fungal host cell is a Mucor miehei cell. In another most particular embodiment, the filamentous fungal host cell is a Myceliophthora thermophilum cell. In another most particular embodiment, the filamentous fungal host cell is a Neurospora crassa cell. In another most particular embodiment, the filamentous fungal host cell is a Penicillium purpurogenum, Penicillium chtysogenum or Penicillium funiculosum (WO 00/68401) cell. In another most particular embodiment, the filamentous fungal host cell is a Thielavia terrestris cell. In another most particular embodiment, the Trichoderma cell is a Trichoderma harzianum, Trichoderma koningii, Trichoderma longibrachiatum, Trichoderma reesei or Trichoderma viride cell.

In a particular embodiment the parent strain is the protease deficient Aspergillus oryzae strain BECh2 described in WO 00/39322, example 1, which is further referring to JaL228 described in WO 98/12300, example 1. This strain, which is alp⁻ and npI⁻ (deficient in the alkaline protease Alp and the neutral metalloprotease NpI) can be further modified to a particularly useful strain according to the invention, in which strain additional mutations have been introduced, as described above, to produce a filamentous fungal strain according to the invention, wherein additionally the serine protease of the subtilisin type designated PepC and/or the calcium dependent, neutral, serine protease, KexB, are deficient.

### Transformation of Filamentous Fungal Host Cells

Filamentous fungal host cells may be transformed by a process involving protoplast formation, transformation of the protoplasts, and regeneration of the cell wall in a manner known per se. Suitable procedures for transformation of Aspergillus host cells are described in EP 238 023, EP 184,438, and Yelton et al., 1984, Proceedings of the National Academy of Sciences USA 81:1470-1474. A suitable method of transforming Fusarium species is described by Malardier et al., 1989, Gene 78:147-156 or in co-pending U.S. Ser. No. 08/269,449.

### Sequence Identity and Alignment

In the present context, the homology between two amino acid sequences or between two nucleic acid sequences is described by the parameter “identity”.

For purposes of the present invention, alignments of sequences and calculation of homology scores may be done using a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6 (W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA”, Methods in Enzymology, 183:63-98).

Multiple alignments of protein sequences may be made using “ClustalW” (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680). Multiple alignments of DNA sequences may be done using the protein alignment as a template, replacing the amino acids with the corresponding codon from the DNA sequence.

Alternatively different software can be used for aligning amino acid sequences and DNA sequences. The alignment of two amino acid sequences is e.g. determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”); e.g. SEQ ID NO: 54 and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap (in the alignment example below this is represented by “|”). The length of a sequence is the number of amino acid residues in the sequence (e.g. the length of SEQ ID NO: 54 is 495).

In the purely hypothetical alignment example below, the overlap is the amino acid sequence “HTWGER-NL” of Sequence 1; or the amino acid sequence “HGWGEDANL” of Sequence 2. In the example a gap is indicated by a “-”.

Hypothetical Alignment Example:

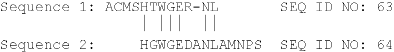

For purposes of the present invention, the degree of identity between two nucleotide sequences is preferably determined by the Wilbur-Lipman method (Wilbur and Lipman, 1983, Proceedings of the National Academy of Science USA 80: 726-730) using the LASER-GENE™ MEGALIGN™ software (DNASTAR, Inc., Madison, Wis.) with an identity table and the following multiple alignment parameters: Gap penalty of 10 and gap length penalty of 10. Pairwise alignment parameters are Ktuple=3, gap penalty=3, and windows=20.

In a particular embodiment, the percentage of identity of an amino acid sequence of a polypeptide with, or to, amino acids 1 to 495 of SEQ ID NO: 54 is determined by i) aligning the two amino acid sequences using the Needle program, with the BLOSUM62 substitution matrix, a gap opening penalty of 10, and a gap extension penalty of 0.5; ii) counting the number of exact matches in the alignment; iii) dividing the number of exact matches by the length of the shortest of the two amino acid sequences, and iv) converting the result of the division of iii) into percentage. The percentage of identity to, or with, other sequences of the invention is calculated in an analogous way.

### Hybridization

For purposes of the present invention, hybridization indicates that the nucleic acid sequence hybridizes to at least one of the nucleic acid sequences shown in SEQ ID NO: 48, 49, 50, or 51 under very low to very high stringency conditions. Molecules to which the nucleotide sequence hybridizes under these conditions may be detected using X-ray film or by any other method known in the art. Whenever the term “polynucleotide probe” is used in the present context, it is to be understood that such a probe contains at least 15 nucleotides.

In one embodiment, the polynucleotide probe is the nucleotide sequence shown in SEQ ID NO: 48, 49, 50, or 51 or the complementary strand of SEQ ID NO: 48, 49, 50, or 51.

In one embodiment hybridization is performed under at least medium stringency conditions, more particularly under at least medium high stringency conditions, and even more particularly under at least high stringency conditions.

For long probes of at least 100 nucleotides in length, very low to very high stringency conditions are defined as pre-hybridization and hybridization at 42° C. in 5×SSPE, 1.0% SDS, 5×Denhardt's solution, 100 μg/ml sheared and denatured salmon sperm DNA, following standard Southern blotting procedures. Preferably, the long probes of at least 100 nucleotides do not contain more than 1000 nucleotides. For long probes of at least 100 nucleotides in length, the carrier material is finally washed three times each for 15 minutes using 2×SSC, 0.1% SDS at 42° C. (very low stringency), preferably washed three times each for 15 minutes using 0.5×SSC, 0.1% SDS at 42° C. (low stringency), more preferably washed three times each for 15 minutes using 0.2×SSC, 0.1% SDS at 42° C. (medium stringency), even more preferably washed three times each for 15 minutes using 0.2×SSC, 0.1% SDS at 55° C. (medium-high stringency), most preferably washed three times each for 15 minutes using 0.1×SSC, 0.1% SDS at 60° C. (high stringency), in particular washed three times each for 15 minutes using 0.1×SSC, 0.1% SDS at 68° C. (very high stringency).

Although not particularly preferred, it is contemplated that shorter probes, e.g. probes which are from about 15 to 99 nucleotides in length, such as from about 15 to about 70 nucleotides in length, may be also be used. For such short probes, stringency conditions are defined as prehybridization, hybridization, and washing post-hybridization at 5° C. to 10° C. below the calculated Tₘ, using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to 99 nucleotides in length, the carrier material is washed once in 6×SCC plus 0.1% SDS for 15 minutes and twice each for 15 minutes using 6×SSC at 5° C. to 10° C. below the calculated Tₘ.

### Materials and Methods

### Materials

### Strains

Aspergillus oryzae NBRC4177 (IFO4177): available from Institute for fermentation, Osaka; 17-25 Juso Hammachi 2-Chome Yodogawa-Ku, Osaka, Japan.
BECh2 is described in WO 00/39322, example 1, which is further referring to JaL228 described in WO 98/12300, example 1.
JaL352 is described in example 7
JaL355 is described in example 7
JaL627 is described in example 10
JaL762 is described in example 17
ICA133 is described in example 7
NZ-17 is described in example 13
NZ-35 is described in example 16
ToC1418 is described in example 7
ToC1510 is described in example 8
ToC1512 is described in example 8

### Genes

pyrG: This gene codes for orotidine-5′-phosphate decarboxylase, an enzyme involved in the biosynthesis of uridine.
HemA: This gene codes for delta-aminolevulinate synthase, an enzyme involved in the biosynthesis of heme.
BIP: This gene codes for a chaperone involved in folding of proteins in the endoplasmic reticulum.

### Plasmids

pUC19: The construction is described in Vieira et al., 1982, Gene 19:259-268
pIC19R: is described in Alting-Mees MA and Short JM, 1989, Nucleic Acids Res, 17: 9494
pA2C315 the plasmid is deposited at DSM under the no. DSM971. The plasmid contains a
cDNA clone from Meripilus giganteus encoding an endoglucanase II gene.
pICA133 is described in example 7D
pJaL240 is described in patent WO 2003008575 example 2
pJaL504 is described in example 9
pJaL574 is described in example 9
pJaL676 is described in WO 03/008575, example 5
pJaL720 is described in example 1
pJaL721 is described in WO 03/008575, example 17
pJaL723 is described in example 1
pJaL728 is described in example 1
pJaL784 is described in example 1
pJaL790 is described in example 1
pJaL800 is described in example 9
pJaL818 is described in example 9
pJaL819 is described in example 9
pJaL835 is described in example 9
pJaL836 is described in example 9
pJaL173 is described in patent WO 98/12300, example 1
pJaL335 is described in patent WO 98/12300, example 1
pJaL680 is described in example 18
pJaL847 is described in example 18
pJaL942 is described in example 18
pDV8 is described in patent WO 01/68864, example 8
pJaL554 is described in patent WO 01/68864, example 8
pMT1623 is described in patent WO 9801470, example 1
pNZ-3 is described in example 2
pNZ-4 is described in example 3
pNZ-6 is described in example 4
pNZa-7 is described in example 5
pNZa-8 is described in example 6
pSO2 is described in patent WO 9735956, example 1
pToC65 is described in patent WO91/17243
pToC381 is described in example 8
pToC465 is described in example 8
pToC466 is described in example 8

### Primers

### Primer B6577F12 (SEQ ID NO: 1)

### Primer B6575F12 (SEQ ID NO: 2)

### Primer H-N (SEQ ID NO:4)

### Primer H-C (SEQ ID NO:5)

### Primer L-N (SEQ ID NO:7)

### Primer L-C (SEQ ID NO:8)

### Primer C315-N (SEQ ID NO:9)

### Primer C315-L-3 (SEQ ID NO:10)

### Primer C315-L-4 (SEQ ID NO:11)

### Primer 8653 (SEQ ID NO:14)

### Primer K1796F04 (SEQ ID NO:15)

### Primer K1796F05 (SEQ ID NO:16)

### Primer K3142D10 (SEQ ID NO:17)

### Primer K3142D11 (SEQ ID NO:20)

### Primer K3142D12 (SEQ ID NO:21)

### Primer K1795F09 (SEQ ID NO:22)

### Primer 104025 (SEQ ID NO: 24)

### Primer 104026 (SEQ ID NO: 25)

### Primer 104027 (SEQ ID NO: 26)

### Primer 104028 (SEQ ID NO: 27)

### Primer 108089 (SEQ ID NO: 28)

### Primer 108091 (SEQ ID NO: 29)

### Primer (SEQ ID NO:30)

### Primer 135944 (SEQ ID NO: 31)

### Primer B2340E06 (SEQ ID NO: 32)

### Primer B2340E07 (SEQ ID NO: 33)

### Primer B2340E08 (SEQ ID NO. 34)

### Primer B2340E09 (SEQ ID NO: 35)

### Primer 101687 (SEQ ID NO: 37)

### Primer 101688 (SEQ ID NO: 38)

### Primer 101689 (SEQ ID NO: 39)

### Primer 101690 (SEQ ID NO: 40)

### Primer 101691 (SEQ ID NO: 41)

### Primer 101692 (SEQ ID NO: 42)

### Primer 172450 (SEQ ID NO: 43)

### Primer 172449 (SEQ ID NO: 44)

### Primer T5483H12 (SEQ ID NO:45)

### Primer T5425G10 (SEQ ID NO:46)

### Primer K4822E06 (SEQ ID NO: 60)

### Primer K4812F11 (SEQ ID NO: 61)

### Methods

General methods of PCR, cloning, ligation of nucleotides etc. are well-known to a person skilled in the art and may for example be found in “Molecular cloning: A laboratory manual”, Sambrook et al. (1989), Cold Spring Harbor lab., Cold Spring Harbor, N.Y.; Ausubel, F. M. et al. (eds.); “Current protocols in Molecular Biology”, John Wiley and Sons, (1995); Harwood, C. R., and Cutting, S. M. (eds.); “DNA Cloning: A Practical Approach, Volumes I and II”, D. N. Glover ed. (1985); “Oligonucleotide Synthesis”, M. J. Gait ed. (1984); “Nucleic Acid Hybridization”, B. D. Hames & S. J. Higgins eds (1985); “A Practical Guide To Molecular Cloning”, B. Perbal, (1984).

### DNA Hybridization

In short all DNA hybridisation was carried out for 16 hours at 65° C. in a standard hybridisation buffer of 10×Denhart's solution, 5×SSC, 0.02 M EDTA, 1% SDS, 0.15 mg/ml polyA RNA and 0.05 mg/ml yeast tRNA. After hybridisation the filters were washed in 2×SSC, 0.1% SDS at 65° C. twice and exposed to X-ray films.

### PCR Amplification

All PCR amplifications were performed in a volume of 100 microL containing 2.5 units Tag po-lymerase, 100 ng of pSO2, 250 nM of each dNTP, and 10 pmol of two of the primers described above in a reaction buffer of 50 mM KCl, 10 mM Tris-HCl pH 8.0, 1.5 mM MgCl2. Amplification was carried out in a Perkin-Elmer Cetus DNA Termal 480, and consisted of one cycle of 3 minutes at 94° C., followed by 25 cycles of 1 minute at 94° C., 30 seconds at 55° C., and 1 minute at 72° C.

### ELISA for Determination of Intact Human IgG

Intact IgG was determined using an ELISA which uses goat anti-human IgG (Fc specific) as the capture antibody and goat anti-human kappa chain conjugated with alkaline phosphatase as the detection antibody. As standard was used a human myeloma IgG1, kappa purified from human plasma. The ELISA procedure was a standard protocol.

### Western Blotting

For Western blotting protein was transferred from SDS-Page gels to membrane filters by Western blotting (Towbin et al., 1979, Proc. Natl. Acad. Sci. USA 76:4350-4354). For detection of heavy chain: The gels were run with a standard protein marker and supernatant from hybridoma cells expressing the same human heavy chain as the A. oryzae cell. Human heavy chain was detected on Western blots by treatment with anti-human IgG (gamma-chain specific) conjugated with alkaline phosphatase (AP) from goat (Sigma A3187) followed by AP color development by incubation with 4-nitro-phenyl phosphate (Sigma N7653) according to the manufacturer's instructions.
For detection of kappa light chain: The gels were run with a standard protein marker and supernatant from hybridoma cells expressing the same human kappa light chain as the A. oryzae cell. Human kappa light chain was detected on Western blots by treatment with anti-human kappa light chain antibody conjugated with alkaline phosphatase (AP) from goat (Sigma A3813) followed by AP color development by incubation with 4-nitro-phenyl phosphate (Sigma N7653) according to the manufacturer's instructions.

### EXAMPLES

### Example 1

### Construction of Aspergillus Expression Plasmid pJaL790

The Aspergillus expression plasmid pJaL790 was constructed in the following way:
The single restriction endonuclease site HindIII in the vector pUC19 was removed by cutting with HindIII and the free overhand-ends was filled out by treatment with Klenow polymerase and the four deoxyribonucleotides and ligated, resulting in plasmid pJaL720. The 1140 by EcoRI-BamHI fragment from pJaL721 was cloned into the corresponding sites in pJaL720, resulting in pJaL723. A 537 by fragment was amplified by PCR with pJaL676 as template and the primers B6577F12 (SEQ ID NO:1) and B6575F12 (SEQ ID NO:2). This was digested with EcoRI, the free overhang-ends were filled in by treatment with Klenow polymerase and the four deoxyribonucleotides and the obtained 524 by fragment was cloned into the HindIII site, which was blunt ended in pJaL723, giving plasmid pJaL728. The single restriction endonuclease site HindIII in the vector pUC19 was removed by cutting with HindIII and the free over-hang-ends were filled in by treatment with Klenow polymerase and the four deoxyribonucleotides and ligated, resulting in plasmid pJaL784. A 1671 by EcoRI-BamHI fragment from pJaL784 was ligated to the 5735 by EcoRI-BamHI fragment from pJaL721, resulting in pJaL790.

### Example 2

### Construction of a Native IgG1 Heavy-Chain Aspergillus Expression Plasmid

A human IgG1 heavy chain encoding sequence was amplified by PCR using SEQ ID NO: 3 as template and the forward primer H-N (SEQ ID NO:4) and the reverse primer H-C (SEQ ID NO:5). Primer H-N and HL-C introduce a BamHI and XhoI restriction site upstream the translational start codon and after the translation termination signal, respectively, for cloning purposes. The PCR product on 1431 by was purified and cut with the restriction endonucleases BamHI and XhoI. The resulting 1419 by fragment was cloned into the corresponding site in pJaL790 to create pNZ-3. DNA from clone pNZ-3 was sequenced to check that it was the right sequence.

### Example 3

### Construction of a Native Kappa Light-Chain Aspergillus Expression Plasmid

A human kappa light-chain encoding sequence was amplified by PCR using SEQ ID NO: 6 as template and with the forward primer L-N (SEQ ID NO:7) and the reverse primer L-C (SEQ ID NO:8). Primer L-N and L-C introduce a BamHI and XhoI restriction site upstream the translational start codon and after the translation termination signal, respectively, for cloning purposes. The PCR product on 732 by was purified and cut with the restriction endonucleases BamHI and XhoI. The resulting 720 by fragment was cloned into the corresponding site in pJaL790 to create pNZ-4. DNA from clone pNZ-4 was sequenced to check that it was the right sequence.

### Example 4

### Construction of Kappa Light-Chain CBD Fusion Aspergillus Expression Vector

A fusion protein between the human kappa light chain used in example 3 and a cellulose binding domain from the endoglucanase II from M. giganteus was constructed by exchanging the DNA sequence encoding the native signal peptide of the light chain with the DNA sequence encoding the M. giganteus cellulose binding domain having its own signal and a linker ending with amino acids KR (CBD) by sequence overlap extension (SOE).
The CBD was amplified by PCR on pA2C315 using the following pair of primers: the forward primer C315-N (SEQ ID NO:9) and the reverse primer C315-L-3 (SEQ ID NO:10). The resulting PCR product on 258 by was purified. The primer C315-N introduces a BamHI restriction site upstream of the translation start codon for cloning purposes.
The light-chain was amplified by PCR starting from pNZ-4 using the following pair of primers: the forward primer C315-L-4 (SEQ ID NO:11) and the reverse primer L-N (SEQ ID NO:7). The resulting PCR product of 671 by was purified.
The two above PCR products were mixed and a standard SOE PCR was preformed with the following pair of primers C315-N and L-N resulting in an amplified fragment of 894 bp. The 894 by fragment was purified and cut with BamHI and XhoI. The resulting 797 by fragment was cloned into the corresponding restriction endonuclease sites of pJaL790 giving an Aspergillus expression plasmid named pNZ-6. The complete amino acid sequence of the CBD fused to the light-chain is given in SEQ ID NO:12.

### Example 5

### Construction of IgG2 Heavy-Chain Aspergillus Expression Plasmid with the IgG1 Heavy Chain Signal Peptide

The signal peptide sequence from the IgG2 heavy chain (SEQ ID NO:13) was exchanged with the native signal peptide of the human IgG1 heavy-chain (SEQ ID NO:3) in the following way: The IgG1 heavy chain signal was amplified by PCR on pNZ-3 using the following pair of primers: the forward primer 8653 (SEQ ID NO:11) and the reverse primer K1796F04 (SEQ ID NO:615). The resulting PCR product of 169 by was digested with BamHI and PvuII and the resulting fragment of 74 by was purified.
The heavy-chain was amplified by PCR using SEQ ID NO:13 as template and the following pair of primers: the forward primer K1796F05 (SEQ ID NO:16) and the reverse primer K3142D10 (SEQ ID NO:17). The resulting PCR product of 1375 by was digested with PvuII and XhoI and the resulting fragment of 1339 by was purified.
The two above fragments were cloned into plasmid pJaL790 digested with BamHI and XhoI resulting in an Aspergillus expression plasmid for the IgG2 heavy chain named pNZa-7. The complete sequence of the IgG1 heavy chain signal fused to the IgG2 heavy-chain is given in SEQ ID NO:18.

### Example 6

### Construction of IgG2 Kappa Light-Chain Aspergillus Expression Plasmid with the IgG1 Kappa Chain Signal Peptide

The signal peptide sequence of the human IgG2 kappa light chain (SEQ ID NO:19) was exchanged with the signal peptide sequence from human kappa light chain (SEQ ID NO:6) by sequence overlap extension (SOE).
The IgG1 kappa signal (SEQ ID NO:6) was amplified by PCR using pNZ-4 as template and the following pair of primers: the forward primer 8653 (SEQ ID NO:14) and the reverse primer K3142D11 (SEQ ID NO:20). The resulting PCR product on 163 by was purified.
The IgG2 kappa chain was amplified by PCR from SEQ ID NO:19 using the following pair of primers: the forward primer K3142D12 (SEQ ID NO:21) and the reverse primer K1795F09 (SEQ ID NO:22). The resulting PCR product of 657 by was purified.
The two above PCR products were mixed and a standard SOE PCR was preformed with the following pair of primers 8653 (SEQ ID NO:14) and K1795F09 (SEQ ID NO:22) resulting in an amplified fragment of 820 bp. The 820 by fragment was purified and restriction digested with BamHI and XhoI. The resulting 723 by fragment was cloned into the corresponding restriction endonuclease sites of pJaL790 giving an Aspergillus expression plasmid named pNZa-8. The complete sequence of the heterologous signal fused to the light-chain is given SEQ ID NO:23.

### Example 7

### Construction of the HemA Minus A. oryzae Strain, ICA133

For removing the defect pyrG gene resident in the alkaline protease gene in the A. oryzae strain BECh2 the following was done:
A. Isolation of a pyrGminus A. oryzae Strain, ToC1418
The A. oryzae strain BECh2 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodiumnitrate as nitrogen source, and 0.5 mg/ml FOA. One strain, ToC1418, was identifying as being pyrGminus. ToC1418 is uridine dependent, therefore it can be transformed with the wild type pyrG gene and transformants selected by the ability to grow in the absence of uridine.
B. Construction of a pyrGplus A. oryzae Strain, JaL352.
The mutation in the defect pyrG gene resident in the alkaline protease gene was determined by sequencing. Chromosomal DNA from A. oryzae strain BECh2 was prepared and by PCR with primers 104025 (SEQ ID NO:24) and 104026 (SEQ ID NO:25) a 933 by fragment was amplified containing the coding region of the defect pyrG gene. The 933 by fragment was purified and sequenced with the following primers: 104025, 104026, 104027 (SEQ ID NO:26), 104028 (SEQ ID NO:27), 108089 (SEQ ID NO:28), and 108091 (SEQ ID NO:29). Sequencing shows that an extra base, a G, was inserted at position 514 in the pyrG-coding region (counting from the A in the start codon of the pyrG gene), thereby creating a frame-shift mutation.
To make a wild type pyrG gene out of the defect pyrG gene resident in the alkaline protease the A. oryzae pyrGminus strain ToC1418 was transformed with 150 pmol of an oligo-nucleotide (SEQ ID NO:30) phosphorylerated in the 5′ end, using standard procedures. The oligo-nucleotide restores the pyrG reading frame, but at the same time a silence mutation is introduce thereby creating a Stul restriction endonuclease site. Transformants were then selected by their ability to grow in the absence of uridine on minimal plates (Cove D. J. 1966. Biochem. Biophys. Acta. 113:51-56) supplemented with 1.0 M sucrose as carbon source, and 10 mM sodiumnitrate as nitrogen source. After reisolation chromosomal DNA was prepared from 8 transformants. To confirm the changes a 785 by fragment was amplified by PCR with the primers 135944 (SEQ ID NO:31) and 108089 (SEQ ID NO:28), which is covering the region of interest. The 785 by fragment was purified and sequenced with the primers 108089 (SEQ ID NO:28) and 135944 (SEQ ID NO:31). One strain having the expected changes was named JaL352.
C. Isolation of a pyrGminus A. oryzae Strain, JaL355
For removing the pyrG gene resident in the alkaline protease gene JaL352 was transformed by standard procedure with the 5.6 kb BamHI fragment of pJaL173 harboring the 5′ and 3′ flanking sequence of the A. oryzae alkaline protease gene. Protoplasts were regenerated on non-selective plates and spores were collected. About 109 spores were screened for resistance to FOA to identify pyrG mutants. After reisolation chromosomal DNA was prepared from 14 FOA resistance transformants. The chromosomal DNA was digested with Bal I and analysed by Southern blotting, using the 1 kb 32P-labelled DNA Bal I fragment from pJaL173 containing part of the 5′ and 3′ flanks of the A. oryzae alkaline protease gene as the probe. Strains of interest were identified by the disappearance of a 4.8 kb Bal I band and the appearance of a 1 kb Bal I band. Probing the same filter with the 3.5 kb 32P-labelled DNA Hind III fragment from pJaL335 containing the A. oryzae pyrG gene showed that the 4.8 kb Bal I band had disappeared in the strains of interest. One strain resulting from these transformants was named JaL355.
D. Construction of a hemA Minus A. oryzae Strain, ICA133.
From the A. oryzae NBRC4177 hemA gene sequence given in U.S. Pat. No. 6,033,892 (Genbank: AF152374), primers were designed to amplify the 5′ flanking and the 3′ flanking sequences. The primers for the 5′ flanking part, B2340E06 (SEQ ID NO:32) and B2340E07 (SEQ ID NO:33) were tailed with BspLU11I and Xho I sites, respectively. The primers for the 3′ flanking part B2340E08 (SEQ ID NO:34) and B2340E09 (SEQ ID NO:35) were tailed with Xho I and Not I sites, respectively.
The amplified fragments on 1068 by and 1153 by were digested with BspLU11I-Xho 1 and Xho I-Not I, resulting in 1049 by fragment and 1132 by fragment, respectively. These fragments were then cloned into BspLU11I-Not I digested pDV8 (vector for positive negative selection). Finally, the pyrG gene of A. oryzae flanked by direct repeats were isolated as a 2346 by Sal I fragment of pJaL554 and inserted into the Xho I site formed between the 5′ and 3′ flanking fragment. The formed plasmid was termed plCA128.
plCA128 was linearized with Not I and used to transform A. oryzae JaL355 and transformants were selected on minimal medium plates supplemented with 250 mM 5′-aminolevulinic acid (5-ALA) and 0.6 mM 5-fluoro-2′-deoxyuridine (FdU) as described in WO 01/68864. A number of transformants were reisolated and plated onto Cove plates without 5-ALA. Two transformants (#2 and #7) growing well on Cove supplemented with 5-ALA, but not growing on Cove without 5-ALA were selected for Southern blot analysis. The chromosomal DNA was digested with Bgl II and analysed by Southern blotting, using the 1049 by 32P-labelled DNA BspLU11I-Xho I fragment from plCA128 containing part of the 5′ flanks of the A. oryzae hemA gene as the probe. Strains of interest were identified by the disappearance of a 1.8 kb Bgl II band and the appearance of a 7.5 kb Bgl II band. The filter was stripped and reprobed with a 476 by 32P-labelled DNA Sal 1-Pst I internal fragment of the A. oryzae hemA encoding part. No hybridization signals are expected if plCA128 integrates by homologous double cross over. For both transformants no hybridization signal was seen. One of the transformants was named ICA133.

### Example 8

### Construction of the pyrG Minus A. oryzae Strain ToC1512

A. Construction of a glaA Minus A. oryzae Strain, ToC1510.
From the A. oryzae NBRC4177 glycoamylase A (glaA) gene sequence (SEQ ID NO:36), primers were designed to amplify the 5′ flanking and the 3′ flanking sequences. The primers for the 5′ flanking part, 101687 (SEQ ID NO:37) and 101688 (SEQ ID NO:38) were tailed with Bgl II and Hind III sites, respectively. The primers for the 3′ flanking part 101689 (SEQ ID NO:39) and 101690 (SEQ ID NO:40) were tailed with Hind III and Sal I sites, respectively. The amplified fragments of 1073 by and 1049 by were digested with Bgl II-Hind III and Hind III-Sal I, resulting in a 1061 by fragment and a 1037 by fragment, respectively. These two fragments were then cloned into Bgl II-Sal I digested pUC19R, resulting in plasmid pToC381. The 2104 by BamHI-Bgl II fragment from pToC381 was blunt ended by treatment with Klenow and the four deoxyribonucleotides and cloned into pDV8 digested with Hind III and blunt ended by treatment with Klenow and the four deoxyribonucleotides giving plasmid pToC465. Finally, the pyrG gene of A. oryzae flanked by direct repeats was isolated as a 2545 by Hind III fragment of pJaL554 and inserted into the Hind III site formed between the 5′ and 3′ flanking fragment. The formed plasmid was termed pToC466.
pToC466 was linearized with Not I and used to transform A. oryzae JaL355 and transformants were selected on minimal medium 0.6 mM 5-fluoro-2′-deoxyuridine (FdU) as described in WO 0168864. A number of transformants were reisolated twice and genomic DNA was prepared. The chromosomal DNA from each of the transformants was digested with Pvu I and analysed by Southern blotting, using the 1061 by 32P-labelled DNA Hind III-Bgl II fragment from pToC381 containing the 5′ flanks of the A. oryzae glaA gene as the probe. Strains of interest were identified by the disappearance of a 1.6 kb Pvu I band and the appearance of a 7.3 kb Pvu I band. The filter was stripped and reprobed with a 1020 by 32P-labelled DNA PCR fragment amplified from A. oryzae genomic DNA by the primers: 101691 (SEQ ID NO:41) and 101692 (SEQ ID NO:42). This PCR fragment encoded part of the A. oryzae glaA protein. No hybridization signals are expected if pToC466 integrates by homologous double cross over, whereas in JaL355 there is a 1.6 kb band. One transformant having the above characteristics was named ToC1510.
B. Isolation of a pyrG Minus A. oryzae Strain, ToC1512
The A. oryzae strain ToC1510 was screened for resistance to 5-fluoro-orotic acid (FOA) to identify spontaneous pyrG mutants on minimal plates (Cove D. J. 1966. Biochem. Biophys. Acta. 113:51-56) supplemented with 1.0 M sucrose as carbon source, 10 mM sodiumnitrate as nitrogen source, and 0.5 mg/ml FOA. One strain, ToC1512, was identified as being pyrG minus. ToC1512 is uridine dependent, therefore it can be transformed with the wild type pyrG gene and transformants selected by the ability to grow in the absence of uridine.

### Example 9

### Construction of the Aspergillus oryzae kexB Deletion Plasmid pJaL836

From plasmid pSO2, which encode the A. oryzae NBRC4177 pyrG gene, a 5336 by SpeI-SspBI fragment and a 316 by Asp718-Nhel fragment (part of the pyrG promoter) were purified and ligated resulting in plasmid pJaL554. The 316 by fragment was cloned down-stream of the encoded pyrG gene, thereby creating a pyrG gene which is flanked by a repeated sequence of 316 bp.

The single restriction endonuclease site BamHI and BglII in pDV8 was removed by two succeeding rounds of cutting with each of the restriction endonucleases and the free overhang-ends were filled in by treatment with Klenow polymerase and the four deoxyribonucleotides and ligated resulting in plasmid pJaL504.

From pJaL504 a 2514 by fragment were amplified by PCR with primer 172450 and 172449 (SEQ ID NO:43 and 44) and cloned into the vector pCR®4Blunt-TOPO resulting in plasmid pJaL574.

From pJaL574 a 2587 by fragment were amplified by PCR with primer T5483H12 and T5425G10 (SEQ ID NO: 45 and 46). This fragment was restriction digested with HindIII and Ndel resulting in a 2582 by fragment, which was cloned into the corresponding site in the vector pUC19 resulting in plasmid pJaL835.

Plasmid pJaL800 contains a 6968 by SalI fragment from A. oryzae NBRC4177 encoding the kexB gene (SEQ ID NO: 47) in pUC19. A 4658 BglII fragment from pJaL800 were subcloned into the BglII site of the vector pIC7 resulting in pJaL818. The repeat flanked pyrG selection marker from pJaL554 were moved as a 2313 by SmaI fragment and cloned into the Ball site of pJaL818 resulting in plasmid pJaL819. The pyrG gene thereby replaces a 911 by Ball encoding part of the kexB gene and the pyrG gene is then flanked by a 1292 by fragment of the 5′ end of kexB and a 2455 by fragment of the 3′ end of kexB. Finally, the deletion cassette of pJaL819 containing the two kexB flanks on either side of the pyrG selection marker was transferred as a 4063 by EcoRI fragment into the EcoRI sites of the TK counter selectable plasmid pJaL835 to give the deletion plasmid pJaL836. Note that pJaL836 contains a unique NotI site immediately downstream of the kexB 5′ flank, which can be used to linearize the plasmid prior to transformation into A. oryzae.

### Example 10

### Construction of a kexB Deleted A. oryzae Strain, JaL627

20 micrograms of pJaL836 was cut with NotI and subsequently the enzyme was heat inactivated as recommended by the manufacturer (New England Biolabs). The plasmid was then ethanol precipitated and re-dissolved in Tris buffer (10 mM pH 8.0) at a concentration suitable for transformation into Aspergillus oryzae.

The linearized plasmid DNA was transformed into Aspergillus oryzae ToC1512 with selection for pyrG and counter selection of the TK gene on FDU plates as previously described in WO 0168864. Transformant colonies were twice re-isolated and finally grown up in liquid medium (YPD). Chromosomal DNA was prepared as previously described in WO 0168864, and used for Southern analysis of the kexB locus with the aim to identify transformants in which a clean double cross-over between the chromosomal kexB and the deletion cassette had occurred. The chromosomal DNA was digested with BglII and BglII-HindIII. The Southern blot was first probed with the 5′ flank excised as a 1292 kb BglII-MluNI fragment from pJaL818 (Probe1). For the wt intact kexB locus, a 4.6 kb BglII fragment is expected to hybridize to this probe for both the BglII and the BglII-HindIII digestion, while for the kexB deleted derivative originating from the desired double cross-over a 6.0 kb fragment and a 1.3 kb fragment will hybridize, respectively. The Southern was stripped of the first 5′ flank probe and re-probed with a probe excised as a 0.8 kb MluNI fragment of pJaL818 (Probe 2). For the wt kexB locus a 4.6 kb fragment was shown to hybridize to this fragment for both digestions, while for the kexB deletion strain originating from the desired cross over no hybridization was obtained. A strain with the above characteristics was preserved as A. oryzae strain JaL627.

### Example 11

### Expression of IgG1 Heavy Chain in Aspergillus oryzae

The strain ICA133 was transformed with the expression plasmid pNZ-3 as described by Christensen et al.; Biotechnology, 1988, 6:1419-1422. In short, A. oryzae mycelia were grown in a rich nutrient broth. The mycelia were separated from the broth by filtration. The enzyme preparation Glucanase 200G® (Novozymes) was added to the mycelia in osmotically stabilizing buffer such as 1.2 M MgSO₄ buffered to pH 5.0 with sodium phosphate. The suspension was incubated for 60 minutes at 37 degrees C. with agitation. The protoplast was filtered through mira-cloth to remove mycelial debris. The protoplast was harvested and washed twice with STC (1.2 M sorbitol, 10 mM CaCl₂, 10 mM Tris-HCl pH 7.5). The protoplasts were finally resuspended in 200-1000 microl STC.
For transformation 5 microg DNA was added to 100 microl protoplast suspension and then 200 microl PEG solution (60% PEG 4000, 10 mM CaCl₂, 10 mM Tris-HCl pH 7.5) was added and the mixture was incubated for 20 minutes at room temperature. The protoplasts were harvested and washed twice with 1.2 M sorbitol. The protoplasts were finally re-suspended 200 microl 1.2 M sorbitol. Transformants containing the amdS gene were selected on minimal plates (Cove D. J. 1966. Biochem. Biophys. Acta. 113:51-56) containing 1.0 M sucrose as carbon source, 10 mM acetamide as nitrogen source, 15 mM CsCl to inhibit background growth, and 250 mM 5-ALA. After 4-5 days of growth at 37 degrees C., stable transformants appeared as vigorously growing and sporulating colonies. Transformants were purified twice through conidiospores.

### Example 12

### Expression of a Kappa Light Chain in Aspergillus oryzae

The strain ToC1512 was transformed with the expression plasmid pNZ-6 as described for the heavy chain in example 12, with the exception that the 250 mM 5-ALA was substituted with 20 mM uridine.
Shake flask containing 10 ml YPM medium (2 g/l yeast extract, 2 g/l peptone, and 2% maltose) was inoculated with spores from the transformants and incubated at 30 degrees C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain.
Transformants which produced the light chain were identified by the appearance of an extra band on 25 kD compared to supernatant from an untransformed parental strain. The identities of the light chain bands were further confirmed by determinations of the N-terminal end by Edman degradation. These data shows that a single dominant sequence of DIQMTQS (SEQ ID NO: 56) was obtained, which corresponds to the human isolated antibody analog.

### Example 13

### Expression of Intact IgG1 Antibody in Aspergillus oryzae Heterokaryons

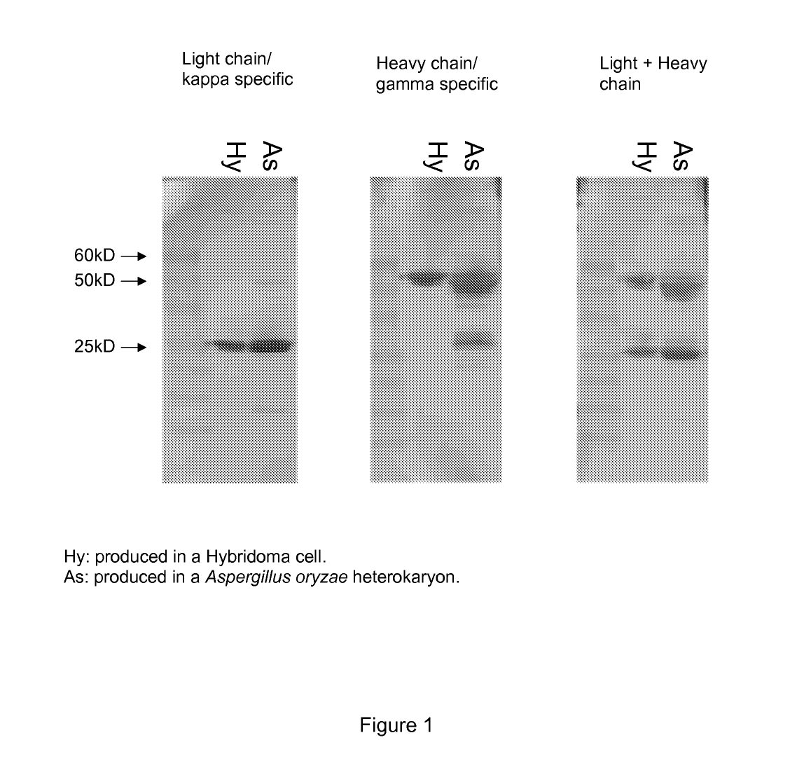

The formation of Aspergillus oryzae heterokaryon cells having mixed nuclei encoding the kappa light chain and the IgG1 heavy chain was done as followed: Approximately 10⁵ spores of a transformant expressing a heavy chain from example 12, which are hemA negative, and of a transformant expressing a light chain from example 13, which are pyrG negative, were mixed in 15 ml COVE media (Cove D. J. 1966. Biochem. Biophys. Acta. 113:51-56) supplemented with 0.02 mM uridine and 25 mM 5′-ALA in a 25 ml NUNC universal container (NUNC 364228). This was incubated for 2 days at 30 degrees C. without shaking. The surface mycelia mats were washed 2 times in sterile water, transferred to COVE plates and incubated 3 days at 37 degrees C. A 1.0 cm square agar plug was transferred to a new COVE plate and incubated 3 days at 37 degrees C. All subsequent handlings of the heterokaryons were done on/in media selecting for heterokaryons.
Shake flask containing 10 ml YPM medium (2 g/l yeast extract, 2 g/l peptone, and 2% maltose) was inoculated with spores from the heterokaryons and incubated at 30 degrees C., 200 rpm for 4 days. The supernatants were analyzed for expression of intact IgG1 by Elisa as described under methods.
From one heterokaryon NZ-17 having the native heavy chain (from example 12) and the CBD light chain fusion (from example 13), the heavy chain associated with the light chain was obtained by protein A chromatography (Goudswaard et al., 1978, Scand J Immunol, 8: 21-28), which is specific for heavy chain. FIG. 1, shows the results of a Western blot using the antibodies described under methods that are specific for the heavy and light chain. The bands observed for the transformant were identified as the heavy chain (50, 53 and 55 kD, probably different glycol forms) and the light chain (25 kD). That the light chain was co-purified with the heavy chain demonstrated that the antibody was assembled.
N-terminal end determination of the bands confirmed that the 3 heavy chain bands had the same sequence, namely EGQLVQSG (SEQ ID NO: 57) and the light chain had the sequence of DIQMTQS (SEQ ID NO: 56), which for both the heavy and light chain corresponds to the sequence of the antibody produced by hybridoma cells.

### Example 14

### Expression of IgG2 Heavy Chain in Aspergillus oryzae

The strain ICA133 (example 7) was transformed with the expression plasmid pNZa-7 as described by Christensen et al.; Biotechnology 1988 6 1419-1422. In short, A. oryzae mycelia were grown in a rich nutrient broth. The mycelia were separated from the broth by filtration. The enzyme preparation Glucanex 200G® (Novozymes) was added to the mycelia in an osmotically stabilizing buffer such as 1.2 M MgSO₄ buffered to pH 5.0 with sodium phosphate. The suspension was incubated for 60 minutes at 37° C. with agitation. The protoplast was filtered through mira-cloth to remove mycelial debris. The protoplast was harvested and washed twice with STC (1.2 M sorbitol, 10 mM CaCl₂, 10 mM Tris-HCl pH 7.5). The protoplasts were finally re-suspended in 200-1000 microl STC.
For transformation 5 microg DNA was added to 100 microl protoplast suspension and then 200 microl PEG solution (60% PEG 4000, 10 mM CaCl₂, 10 mM Tris-HCl pH 7.5) was added and the mixture is incubated for 20 minutes at room temperature. The protoplasts were harvested and washed twice with 1.2 M sorbitol. The protoplasts were finally re-suspended in 200 microl 1.2 M sorbitol. Transformants containing the amdS gene were selected on minimal plates (Cove D. J. 1966. Biochem. Biophys. Acta. 113:51-56) containing 1.0 M sucrose as carbon source, 10 mM acetamide as nitrogen source, 15 mM CsCl to inhibit background growth, and 250 mM 5-ALA. After 4-5 days of growth at 37° C., stable transformants appeared as vigorously growing and sporulating colonies. Transformants were purified twice through conidiospores.

### Example 15

### Expression of a Kappa Light Chain in Aspergillus oryzae

The strain ToC1512 (example 8) was transformed with the expression plasmid pNZa-8 as described for the heavy chain in example 14, with the exception that the 250 mM 5-ALA was substituted with 20 mM uridine.
Shake flask containing 10 ml YPM medium (2 g/l yeast extract, 2 g/l peptone, and 2% maltose) was inoculated with spores from the transformants and incubated at 30 degrees C., 200 rpm for 4 days. Samples of supernatant (20 microl) were mixed with an appropriate volume of 2× sample loading buffer and subjected to sodium dodecyl sulfate polyacrylamide gel electrophoresis (SDS-PAGE) according to the manufacturer's instructions (Novex NuPAGE 10% Bis-Tris Electrophoresis System from Invitrogen Corporation). The gels were stained for protein with Coomassie Brilliant Blue stain or the protein was transferred to membrane filters by Western blotting (Towbin et al., 1979, Proc. Natl. Acad. Sci. USA 76:4350-4354). Human kappa light chain was detected on Western blots by treatment with anti-human kappa light chain antibody conjugated with alkaline phosphatase (AP) from goat (Sigma A3813) followed by AP color development by incubation with 4-nitro-phenyl phosphate (Sigma N7653) according to the manufacturer's instructions. A Western blot of the light chain is shown in FIG. 3, third gel.
Transformants which produced the light chain were identified by the appearance of an extra band on 25 kD compared to supernatant from an untransformed parental strain. The identity of the light chain bands were further confirmed by determinations of the N-terminal end by Edman degradation. These data shows that a single dominant sequence of EIVLTQS (SEQ ID NO: 58) was obtained for all 4 expression constructs, which corresponds to the human isolated antibody analog.

### Example 16

### Expression of Intact IgG2 Antibody in Aspergillus oryzae Heterokaryons

The formation of Aspergillus oryzae heterokaryon cells having mixed nuclei encoding the kappa light chain and the IgG2 heavy chain was done as followed: Approximately 10⁵ spores of a transformant expressing a heavy chain from example 14, which are hemA negative, and of a transformant expressing a light chain from example 15, which are pyrG negative, were mixed in 15 ml COVE media (Cove D. J. 1966. Biochem. Biophys. Acta. 113:51-56) supplemented with 0.02 mM uridine and 25 mM 5′-ALA in a 25 ml NUNC universal container (NUNC 364228). This was incubated for 2 days at 30 degrees C. without shaking. The surface mycelia mats were washed 2 times in sterile water, transferred to COVE plates and incubated 3 days at 37 degrees C. A 1.0 cm square agar plug was transferred to a new COVE plate and incubated 3 days at 37 degrees C. All subsequent handlings of the heterokaryons were done on/in media selecting for heterokaryons.
Shake flask containing 10 ml YPM medium (2 g/l yeast extract, 2 g/l peptone, and 2% maltose) was inoculated with spores from the heterokaryons and incubated at 30 degrees C., 200 rpm for 4 days. The supernatants were analyzed for expression of intact IgG2 by Elisa as described in methods.
From one heterokaryon NZ-35 the heavy chain associated with the light chain was obtained by protein A chromatography (Goudswaard et al., 1978, Scand J Immunol, 8: 21-28), which is specific for heavy chain. FIG. 3 shows gels of the results of a Western blot using the antibodies described in methods that are specific for the heavy and light chain. The bands observed for the transformant were identified as the heavy chain (50, and 55 kD, probably different glycol forms) and the light chain (25 kD). The fact that the light chain was co-purified with the heavy chain demonstrated that the antibody was assembled.
N-terminal end determination of the bands confirmed that the 2 heavy chain bands had the same sequence, namely EVQLLQSG (SEQ ID NO: 59) and the light chain had the sequence of EIVLTQS (SEQ ID NO: 58), which for both the heavy and light chain corresponds to the sequence of the antibody produced by CHO cells.

### Example 17

### Expression of the IgG1 Antibody in A. oryzae JaL627

The strain JaL627 was transformed with both expression plasmids pNZ-3, and -4 as described in example 11 and a transformant expressing intact IgG1 were selected as described in example 13 and was named JaL762

### Example 18

### Construction of BIP Expression Plasmid pJaL942

The plasmid for expression of BIP was done by the following: The 2045 by EcoRI-XbaI fragment containing the BAR expression cassette from pMT1623 was cloned into the corresponding restriction site in vector pToC65 giving plasmid pJaL680. Plasmid pJaL680 was digested with BstXI and EcoRI and the ends was blunted by filling in by adding the Klenow fragment and 4 dNTP's, and the resulting 4729 by fragment was purified from an agarose gel and re-ligated giving plasmid pJaL847.

The A. oryzae BIP gene (SEQ ID NO: 62) was amplified by PCR with the primers K4822E06 (SEQ ID NO: 60) and K4812F11 (SEQ ID NO: 61) using A. oryzae NBRC4177 genomic DNA as template. This gave a 2407 by fragment which was digested with BamHI and EcoRI to give a 2395 by fragment containing the coding region of the BIP and 201 by down stream of the BIP coding region containing the terminator.

The A. niger neutral amylase 2 (NA2) promoter was purified as a 611 by EcoRI-BamHI fragment from pJaL240. The 2395 by and 611 by fragments was cloned into the unique EcoRI site of pJaL847 giving plasmid pJaL942. This plasmid then contained the A. oryzae BIP gene under the A. niger neutral amylase 2 promoter control and the BAR gene under the A. oryzae TPI promoter control which is used as a marker for transformation of Aspergillus strains.

### Example 19

### Overexperssion of BIP in Antibody Producing Aspergillus Strains

The strains NZ-17 (example 13), NZ-35 (example 16) and JaL762 (example 17) were trans-formed with plasmid pJaL942. The transformation procedure is described in EP Application No. 87103806.3. Transformants were selected by resistance to 1 mg/ml glufosinate in minimal plates (Cove D. J. (1966) BBA113 51-56) containing 1 M sucrose for osmotic stabilization and 10 mM (NH₄)₂SO₄. All transformants from each strain were spore isolated twice, grown in 10 ml YPM and intact antibody yields were determined as described in example 13. Table 1, 2 and 3 shows the results obtained for the different transformants obtained by transformation of pJaL942 into the 3 strains NZ-17, NZ-35 and JaL762, respectively.

The results shown in the tables below show that transformants can be obtained which result in an increased yield of the secreted antibody from a factor 1.5 up to a factor 9 compared to the untransformed strains.

TABLE 1

| Transformant no. | Yields (ng/L) |
|---|---|
| 2 | 2200 |
| 3 | 1600 |
| 4 | 1000 |
| 5 | 2000 |
| 6 | 1500 |
| 7 | 2400 |
| 8 | 1800 |
| 9 | 2500 |
| 10 | 500 |
| 11 | 400 |
| 12 | 5400 |
| 13 | 1200 |
| 14 | 1800 |
| 15 | 600 |
| 16 | 4300 |
| 17 | 3200 |
| 18 | 1400 |
| 19 | 1200 |
| 20 | 1600 |
| NZ-17 | 1600 |

TABEL 2

| Transformant no | Yields (ng/ml) |
|---|---|
| 1 | 1741 |
| 2 | 100 |
| 3 | 600 |
| 4 | 100 |
| 5 | 800 |
| 6 | 3059 |
| 7 | 10849 |
| 8 | 200 |
| NZ-35 | 1195 |

TABEL 3

| Transformant no | Yields (ng/L) |
|---|---|
| 1 | 8621 |
| 2 | 8698 |
| 3 | 11491 |
| 4 | 10673 |
| 5 | 11952 |
| 6 | 11482 |
| 7 | 9921 |
| 8 | 10069 |
| 9 | 7218 |
| 10 | 6188 |
| 11 | 3549 |
| 12 | 4446 |
| 13 | 1622 |
| 14 | 9431 |
| 15 | 10299 |
| 16 | 10197 |
| 17 | 9780 |
| 18 | 12051 |
| 19 | 7118 |
| 20 | 7720 |
| 21 | 6491 |
| 22 | 7770 |
| 23 | 9668 |
| 24 | 7774 |
| 25 | 5887 |
| 26 | 10035 |
| 27 | 8848 |
| 28 | 6807 |
| 29 | 6886 |
| 30 | 6192 |
| 31 | 9952 |
| 32 | 6192 |
| 33 | 5532 |
| 34 | 8324 |
| 35 | 11086 |
| JaL762 | 8405 |

1. A method for increasing the production yield of a secreted antibody or antibody fragment in a filamentous fungal host cell, comprising: recombinant expression of the antibody or antibody fragment and over-expression of a BiP chaperone protein.

2. The method according to claim 1, wherein the BiP chaperone is an Aspergillus BiP protein.

3. The method according to claim 2, wherein the BiP chaperone protein is BiPA from Aspergillus oryzae.

4. The method according to claim 1, wherein the BiP protein expression is controlled by a promoter selected from the group consisting of A. oryzae TAKA amylase, NA2, NA2-tpi, glaA, tpi, gpd, tef1, and pgkA promoters.

5. The method according to claim 1, wherein the antibody or antibody fragment is selected from the group consisting of IgG1, IgG2, IgG3, IgG4, IgM, IgA, IgD, IgE, F(ab′)₂, and Fab.

6. The method according to claim 1, wherein the antibody is an IgG antibody.

7. The method according to claim 1, wherein the filamentous fungal host cell has a reduced or no expression of one or more proteases selected from the group consisting of PepC, KexB, Alp, NpI.

8. The method according to claim 1, wherein the filamentous fungal host cell is a heterokaryon.

9. The method according to claim 1, wherein the filamentous fungal cell is selected from the group consisting of Acremonium, Aspergillus, Fusarium, Humicola, Mucor, Myceliophthora, Neurospora, Penicillium, Thielavia, Tolypocladium, or Trichoderma.

10. The method according to claim 9, wherein the Aspergillus cell is selected from the group consisting of Aspergillus awamori, Aspergillus foetidus, Aspergillus japonicus, Aspergillus nidulans, Aspergillus niger or Aspergillus oryzae.

11. The method according to claim 10, wherein the Aspergillus cell is A. oryzae.

12. The method according to claim 10, wherein the Aspergillus cell is A. niger.
